(19) United States
(12) Patent Application Publication
(10) Pub. No.: US 2024/0076601 A1
(43) Pub. Date: Mar. 7, 2024

(54) INCUBATOR AND METHOD

(71) Applicant: EPPENDORF AG, Hamburg (DE)

(72) Inventors: Alexandra Jankowski, Hamburg (DE); Philipp Kayser, Hamburg (DE); Wolf Lukas Hellweg, Hamburg (DE); Niklas Lehrke, Hamburg (DE)

(21) Appl. No.: 18/262,150

(22) PCT Filed: Jan. 27, 2022
(86) PCT No.: PCT/EP2022/051876

§ 371 Date: Jul. 19, 2023

(30) Foreign Application Priority Data
Jan. 27, 2021 (EP) 21153810.3

(51) Int. Cl. C12M 1/00; C12M 1/36; C12M 3/06

(52) U.S. Cl. CPC C12M 41/14; C12M 27/16; C12M 41/06; C12M 41/48

(57) ABSTRACT

The invention relates to a live cell culture incubator, a method of working with an incubator, and a system including an incubator. An image acquisition system is used to determine an occupancy of the incubator.

The invention relates to a live cell culture incubator, a method of working with an incubator, and a system comprising an incubator.

Such incubators are used in biological and medical laboratories to maintain cells in cell culture under controlled environmental conditions, thus enabling the growth of live cells in vitro. For this purpose, the temperature and gas composition or humidity of the atmosphere inside an incubator chamber isolated from the environment are maintained at the desired values by the incubator's apparatus. Eukaryotic cells require CO₂ incubators. The atmosphere is formed by air with a certain CO₂ and O₂ content and a certain humidity, a suitable temperature is often 37° C.

Cell growth is particularly critically dependent on the constancy of atmospheric conditions in the incubator. Disturbances of the incubator atmosphere can have a negative effect on the growth of the cells. In an “ideally” equipped laboratory, each individual user would be provided with a separately accessible incubation chamber for each sample to be incubated. However, this is not realistic for reasons of cost efficiency. In laboratory practice, it is common for a single incubator (or a few incubators) with a single incubation chamber and one or more storage areas on one or more storage plates in the incubation chamber to be provided for use by multiple users.

According to the studies underlying the present invention, the frequency of opening the chamber door of the incubator and thus interfering with the controlled atmosphere of the incubator scales with the number of users and, moreover, with the number of samples incubated there. The intensity of the interference also depends on the duration of the door opening. The more time a user needs to access the interior space of the incubator chamber, the longer the door remains open.

There are several incubator usage scenarios that may require increased access time due to complications:

Scenario A) Setting New Objects in the Incubator.

When a user places one or more objects, especially cell culture containers, into the incubator, he needs a free storage space in a storage area. If no free storage space is available due to disordered storage, the user needs time to create this storage space; the more carefully he moves or rearranges objects already present in the incubator (inventory objects), possibly even documenting this in writing, the more time-consuming the process becomes. If it turns out that there is no longer sufficient storage space available in the incubator chamber, the user repeats his procedure for another compartment of the incubator chamber or for a possible replacement incubator of the laboratory. This extends the period of the condition of an open incubator door, i.e. the duration of exposure of the chamber interior space to the environment (exposure duration).

Scenario B) Testing Cell Cultures

If a user tests the cell cultures he/she has previously placed in the incubator, e.g. to assess the quality of the cell medium or the state of growth, the user will first search for the cell culture container in question in the incubator. This will extend the exposure time. The more carefully the user moves or rearranges stock objects in the process, the more time-consuming the search process becomes.

Scenario C) Removing the Objects from the Incubator

In this case, too, the user must first search for the corresponding object. The time-delaying factors mentioned in B) apply.

Moreover, the more frequently an incubator is opened, the higher the risk of contamination of the interior space. There are also cases, for example in forensics or reproductive medicine, where the value of a single sample, especially cell(s) contained in a cell culture vessel, is much higher than, for example, the value of the entire incubator, so that loss of the sample due to contamination must be avoided at all costs. In any case, the frequency of contamination increases the risk of work stoppage, increases costs, and demands additional maintenance. After contamination of an incubator, the chamber must be cleaned and sterilized before the incubator can be used further. During this time, unless a replacement incubator is available, work with cell cultures is interrupted.

In laboratories, therefore, there is a fundamental need to keep the period during which the incubator door is open as short as possible and also to keep the frequency of opening the incubator chamber as low as possible.

The task underlying the present invention is to provide an incubator which can be used efficiently and, in particular, which makes it possible to keep the exposure period, i.e. the period of the state of an open incubator door, and thus the period of exposure of the interior space of the incubator chamber to the environment of the incubator low.

The invention solves this problem by the incubator according to claim 1, the system according to claim 16, and the method according to claim 17. Preferred embodiments are in particular objects of the dependent claims.

The incubator according to the invention for incubating live cell cultures, comprises:

Incubator for incubating live cell cultures, comprising
an incubator chamber for receiving objects, in particular cell culture containers, which comprises opposing inner walls and a chamber opening for the feeding and removal of the objects by a user, and which comprises at least one storage area for storing the objects extending between the opposing inner walls,
an incubator door to close the chamber opening,
an image acquisition system comprising
   an illumination device,
   at least one camera device and
   a data processing device with a data memory,
      wherein the image acquisition system is configured to, in particular with the incubator door closed or open,
   the illumination device is used to illuminate the storage area extending between the inner walls,
   capturing, by means of the camera device, at least one image of the storage area extending between the inner walls, and
   storing the at least one image by means of the data processing device in the form of image data in the data storage device.

The image acquisition system makes it possible to take images in the incubator chamber under controlled conditions and in a reproducible manner, which can provide correspondingly versatile information about the occupancy of the storage area. Since the incubator according to the invention provides information about the occupancy of the storage area in the form of image data, users are enabled to retrieve information about the occupancy status of the incubator before opening the incubator. By providing this information, unnecessary opening of the incubator is reduced and use of the incubator becomes more efficient.

The processing of this information about the occupancy of the storage area can consist of providing the user with an image of the storage area, for example by displaying it on a display of the incubator. In this way, the user can immediately get a “picture” of whether the occupancy of the storage area permits the setting of further objects, and whether the corresponding storage area contains an object set by him and now to be checked or removed—insofar as it is identifiable for the user—and where this could be found. Furthermore, the information can also be processed further by using automated image evaluation to obtain data on free storage space in this storage area and communicate this to the user. Also a recognition of object classes and object individual characteristics is made possible by the availability of the image data.

The incubator is a laboratory device or a laboratory incubator. In particular, an incubator refers to a laboratory device with an incubator chamber whose atmosphere can be controlled or regulated by the incubator to a predetermined target temperature. In particular, it is a laboratory device, with which controlled climatic conditions for various biological development and growth processes can be created and maintained. The incubator may be or include a shaker incubator, i.e., an incubator with a motion device for moving objects placed in the incubator chamber, and may be a microbial incubator (also without CO₂). The incubator may in particular be designed as a cell cultivation device. In particular, the incubator serves to create and maintain a microclimate with controlled gas, and/or humidity, and/or temperature conditions in the incubator chamber, which treatment may be time-dependent. The laboratory incubator, in particular a treatment device of the laboratory incubator, may in particular comprise a timer, in particular a timer, a heating/cooling device and preferably an adjustment for the regulation of an exchange gas supplied to the incubator chamber, an adjustment device for the composition of the gas in the incubator chamber of the incubator, in particular for adjusting the CO₂ and/or the O₂ and/or the N₂ content of the gas and/or an adjustment device for adjusting the humidity in the incubator chamber of the incubator. The incubator, in particular a treatment device of the incubator, has in particular the incubator chamber, further preferably a control device with at least one control loop, to which the at least one heating/cooling device is assigned as an actuator and at least one temperature measuring device is assigned as a measuring element. By means of the control device, the temperature in the incubator can be controlled. Depending on the embodiment, the humidity can also be controlled via it. A tub filled with water in the incubator chamber can be heated or cooled in order to adjust the humidity via evaporation. Alternatively and/or additionally, a water evaporator can be provided as part of the incubator, by means of which the humidity in the atmosphere of the incubator chamber is adjusted. CO₂ incubators are used in particular for the cultivation of animal or human cells. Incubators may comprise turning devices for turning the at least one cell culture container and/or a shaking device for shaking or moving the at least one cell culture container. The incubator according to the invention is in particular not a bioreactor or fermentor.

The incubator may comprise a sensor device. In particular, a sensor device comprises at least one temperature sensor, preferably a plurality of temperature sensors. For example, a temperature sensor may be a Pt 100 or Pt 1000 temperature sensor. A sensor device preferably has a sensor for determining a relative gas concentration, in particular for determining the content of CO₂ and/or O₂ and/or N₂. A sensor device preferably has a sensor for determining the relative humidity of the air.

An incubator preferably comprises one or a single incubator chamber. This may be divided into compartments. Compartments may be separated by—in particular perforated—bearing plates, whereby in particular a gas exchange between the compartments is made possible. A bearing plate, in particular its lower side, may be set up to hold the camera device and can in particular have a holder for the camera device. A bearing plate, in particular the lower side thereof, may be arranged for holding the illumination device and may in particular comprise a holder for the illumination device. However, the illumination device or its holder may also be arranged or mounted elsewhere in the incubator chamber, for example on an inner side wall of the incubator chamber, or on the floor or ceiling wall. A fixture for the illumination device may include a rail system, a robotic arm controlled by the control device, and/or magnet(s).

The incubator chamber comprises chamber walls or chamber inner walls and exactly one or at least one chamber opening via which the objects or cell culture containers can be placed inside the incubator chamber and removed. This chamber opening is closable by a closure element movably connected to the incubator chamber, in particular an incubator door movably mounted on the incubator chamber by means of a hinge, in particular one or more chamber doors. An incubator can have one or more inner doors, which can in particular be transparent, and can have an—in particular non-transparent—outer door, which in particular thermally insulates the incubator chamber and, if applicable, at least one inner incubator door, which closes or opens the chamber opening, from the environment. Preferably, images are captured by the image acquisition system when the incubator door or outer door is closed, so that ambient light does not influence the illumination of the storage area, which is preferably performed exclusively by the illumination device. This leads to particularly well reproducible image recordings that can be easily compared and evaluated by image processing algorithms. Nevertheless, it is also possible for the image recordings to be created with the incubator door open.

In the closed position of the chamber opening, the interior space of the incubator chamber is preferably insulated from the environment in such a way that a desired temperature or atmosphere controlled by the incubator can be set, in particular regulated, in the interior space. In the open position of the chamber opening, gas exchange between the environment of the incubator and the interior space of the incubator chamber is possible via this opening. The chamber opening is typically located in a front wall surrounding the chamber opening.

The incubator chamber preferably has a plurality of walls or inner wall surfaces which can be connected to one another, in particular integrally and in particular without edges. The walls or inner wall surfaces are preferably substantially planar in shape, but may also all or in part have a curved shape. The incubator chamber is preferably cuboidal in shape, but may also be otherwise shaped, e.g. spherical, ellipsoidal, polyhedral. The walls or inner wall surfaces are preferably made of a low-corrosion material, in particular stainless steel, copper, brass, or a plastic, in particular a composite plastic. This facilitates cleaning/disinfection of the chamber interior space. Independently of the chamber opening, which serves to load/unload objects or cell culture containers, the incubator chamber can have at least one port for passing an appropriately dimensioned device or cable connection from the interior space of the incubator chamber to its exterior or to the environment of the incubator.

Preferably, the surfaces of the incubator inner walls are designed to be non-glossy or non-reflective, in particular by using a matte surface. The surface of the incubator inner wall can be matted by a surface treatment. The surface treatment may in particular be grinding with an abrasive, which may in particular have a specific grain size. The surface treatment can in particular be irradiation with a blasting medium, in particular sand or glass beads, in particular by compressed air, which can in particular have a specific grain size or a characteristic particle diameter. This can prevent or reduce disturbing reflections in an image.

A typical size of the interior space of an incubator chamber is between 50 and 400 liters.

The incubator may comprise exactly one incubator chamber, but can also have several incubator chambers, the atmosphere of which (temperature, relative gas concentration, humidity) can be adjustable, in particular individually or collectively. An incubator can have several incubator chambers, each of which can have its own chamber opening and its own chamber door for closing the chamber opening.

The incubator may comprise a housing that partially or completely surrounds the incubator chamber. The housing may be substantially cuboidal in shape, and may in particular be designed such that the incubator is stackable.

A storage area of the incubator is realized in particular by a storage plate, in particular a shelf plate insert and/or a moving platform, which in particular can be made of stainless steel or copper or the like or have this material. A bearing plate serves as a bottom plate, in particular as an intermediate bottom plate. The bearing plate can be removable from the incubator chamber (“bearing plate insert”) or can be permanently connected to it. The incubator chamber may have holding sections or a holding frame for holding one or more bearing plate inserts or insertable instruments. A bearing plate may be arranged on its underside for holding a camera, in particular have a holder for this camera. Alternatively or additionally, at least one of the inner walls of the incubator chamber may be arranged for holding one or more bearing plate inserts or insertable instruments, in particular the at least one camera. For this purpose, a holding structure integrated into the wall may be provided, in particular one or more protrusions, grooves or webs. A storage plate increases the available storage area in the incubator chamber.

Preferably, substantially all surfaces or at least one surface of the at least one bearing plate are designed to be non-glossy or non-reflective, in particular by using a matte surface. The surface of the incubator inner wall may be matted by a surface treatment. The surface treatment may in particular be grinding with an abrasive, which may in particular have a specific grain size. The surface treatment can in particular be irradiation with a blasting medium, in particular sand or glass beads, in particular by compressed air, which can in particular have a specific grain size or a characteristic particle diameter. This can prevent or reduce disturbing reflections in an image.

A holding frame for the at least one bearing plate is also preferably made of a non-corrosive material, preferably stainless steel. The holding frame is preferably designed as a standing object by having at least one base section that rests on the bottom wall of the incubator chamber. However, it may also be supported on the side walls of the incubator chamber and/or suspended from the ceiling wall of the incubator chamber.

A bearing plate preferably—and in particular substantially completely—extends across a horizontal cross-section of the incubator chamber.

Preferably, an incubator comprises at least two storage plates arranged one above the other. The volume area between two bearing plates, or between a bottom wall of the incubator chamber and a lowermost bearing plate or between a top wall of the incubator chamber and an uppermost bearing plate can be referred to as a storage compartment. A storage compartment may be construed as a whole as a storage area. The surface of a storage plate suitable for storage can be understood as a storage area. The height of a storage compartment is preferably dimensioned such that an object of a certain maximum height (measured perpendicular to the planar surface of a storage plate) or an object stack of objects of a certain maximum height of the stack can be placed on the storage plate. In particular, the maximum height can essentially correspond to the distance between two bearing plates.

The distance between two bearing plates or the maximum height is in particular between 5 cm and 70 cm, preferably between 5 cm and 65 cm, preferably between 5 cm and 60 cm, preferably between 5 cm and 50 cm, preferably between 10 cm and 30 cm, preferably between 10 cm and 20 cm, preferably between 12 cm and 18 cm. In particular, the maximum height can be up to 150 cm. The distance between two bearing plates is preferably selectable by the user by means of a variable holding device for bearing plates.

An instrument that may be inserted into the interior space of the incubator chamber, in particular a camera, can be designed as a module and enables automated observations to be made inside, preferably even when the incubator door is closed.

The camera device, or its at least one camera, is preferably arranged on a bearing plate, preferably arranged or arrangeable below a bearing plate and in particular fastened or mountable thereon. Preferably, at least one camera is mounted or mountable on the underside of a bearing plate, in particular in a geometric center of the underside, in particular in the intersection of the diagonals of a rectangular underside.

Preferably, one camera—or several cameras—is mounted or mountable on the underside of a shelf insert in the incubator chamber, or on an underside of the upper inner wall (ceiling wall) of the incubator chamber, preferably vertically above the geometric center of the storage area monitored by the camera. However, one or more cameras can also be arranged or mounted or arranged/mounted on inner side walls of the incubator chamber or on a holding frame.

Preferably, the at least one camera is configured and arranged to have an angle of view, typically measured in the image diagonal or, alternatively, in the image vertical or image horizontal, that is between 90° and 210°, preferably between 120° and 180°, and more preferably between 160° and 180°.

A camera of the image acquisition system can have wide-angle optics, in particular fisheye optics, whose image angle in the image diagonal can be between 120° and 230°.

Preferably, exactly one camera is provided on the underside of a bearing plate, which in particular comprises one of the angles of view mentioned above.

The “field of view” or field of view (FOV) of a camera can be defined in particular as having a certain image angle, e.g. an image angle according to one of the ranges defined above, which leads to the representation of an image content dependent on this angle of view, or can be defined by an image angle measured in the image vertical and an image angle measured in the image horizontal. In particular, the aspect ratio of the image may be one of the following formats: 4:3, 3:2, 16:9, 1:1.

Preferably, at least one camera—preferably exactly one camera—is arranged on the underside of a bearing plate, the viewing area of which preferably covers more than X % of the bearing surface of a bearing plate lying in the viewing area of the at least one camera. X is, in each case preferably 20, 30, 40, 50, 60, 70, 80, 90, 100. In other words: the—exactly one or at least one—image recorded by this—exactly one or at least one—camera preferably shows more than X % of the storage area of a storage plate lying in the viewing area of the at least one camera. For example, several cameras can be provided which together capture the entire storage area, i.e. 100% of the storage area, or the proportion X. Preferably, exactly one camera is provided that captures the entire storage area, or the portion X. The larger or more complete the field of view, the more reliably or efficiently the storage area can be imaged and the image evaluated.

Preferably, at least one camera—preferably exactly one camera—is arranged on the underside of a storage plate, the viewing area of which camera is located in a compartment of the incubator and which, preferably in addition to the storage area (a part or the entire storage area) on the storage plate, preferably captures more than Y % of the wall area of a compartment wall bounding the compartment, which compartment wall is formed by an inner wall section of the inner wall of the incubator. Y is, respectively, preferably 20, 30, 40, 50, 60, 70, 80, 90, 100. In other words, that—exactly one or at least one—image captured by that—exactly one or at least one—camera preferably shows more than Y % of the wall area of one (or all) compartment wall bounding the compartment. For example, several cameras may be provided which together capture the entire area of all compartment walls, i.e. 100% of the interior space wall area of a compartment, or the portion Y. Preferably, exactly one camera is provided that captures the entire area of all compartment walls, or portion Y. Due to the correspondingly large field of view, objects or object stacks positioned at an edge of the planar storage area of the storage plate can also be detected in particular.

The data processing device is preferably programmed to automatically crop the image captured by the camera to produce an effective angle of view smaller than the angle of view specifying the camera, or to produce an effective field of view smaller than the field of view specifying the camera.

The incubator camera is particularly suitable to work reliably in the respective incubator atmosphere over a period of several months or years, or to work reliably during the lifetime measured under standard conditions (room temperature). Not every camera is suitable to operate in an incubator atmosphere. One possible commercially available camera is the 5MP Wide Angle Camera for Raspberry Pi, www.joy-it.net, available from Conrad Electronic SE, Germany, and/or another camera in combination with a wide angle lens, e.g. commercially available the “Industrial lens HAL 250 2.3”, Entaniya Co., Ltd., Japan. Alternatively, a cover device may be provided for the at least one camera to shield or isolate it from the incubator atmosphere, said cover device in particular having transparent regions or a transparent window or being transparent to allow image capture through the transparent regions.

Preferably, the camera device comprises at least one optical filter with which the light incident on the camera is filtered. This allows the quality of the image recording to be optimized, in particular with regard to downstream digital image processing and evaluation. Preferably, the camera device has at least one polarizing filter with which the light incident on the camera is filtered. This makes it possible to reduce reflections in the image recording that can arise from reflections of the light from the illumination device on objects in the storage area, elements of the storage area or the incubator chamber and/or inner walls of the incubator chamber, in particular with regard to downstream digital image processing and evaluation. The polarizing filter is preferably a circular polarizing filter, but can also be linear. Unwanted reflections from smooth, non-metallic surfaces (e.g. the plastic surface of cell culture containers) can be suppressed by means of polarizing filters. On non-metallic surfaces, light with perpendicular polarization is reflected noticeably more, especially if the exit angle to the surface is about 30° to 40°, i.e. close to the Brewster angle. If the polarizing filter is suitably oriented, the reflected light waves are suppressed so that the unpolarized background is not outshone by the reflections. Preferably, when polarization is used, the objects to be imaged, in particular cell culture containers, are arranged—in particular in a direct line—between the illumination device and the at least one camera.

Preferably, the camera device comprises a first polarizing filter and the illumination device has a second polarizing filter, wherein in particular the first and second polarizing filters are used rotated relative to each other. In this way, part of the light from the illumination device is initially blocked out by the polarizing filter in front of it. The polarizing filter in front of the camera is adjusted or rotated with respect to the polarizing filter of the illumination device in such a way that it now also blocks out the other part of the light emitted by the illumination device. Ideally, only diffuse light remains. As a result, reflections, even from metallic surfaces, are softened or completely eliminated. This is particularly advantageous with regard to downstream digital image processing and evaluation, especially with regard to outline recognition of objects by means of image processing.

Preferably, the illumination device comprises at least one light source, in particular LED. Preferably, the illumination device has at least two light sources or more, each with a different emission spectrum, i.e. different colors, e.g. red, green, blue. In this way, the image quality can be optimized, particularly with regard to downstream digital image processing and evaluation, especially with regard to outline recognition of objects by means of image processing.

The illumination device can comprise at least one light source whose emitted light has wavelengths greater than that of visible light or consists thereof, in particular whose emission spectrum lies in the infrared range with, in particular, a wavelength between 780 nm and 1 mm, in particular in the near infrared (780 nm to 3000 nm) or mid infrared (3000 nm to 50000 nm) or contains such an infrared range. In this case, the camera device comprises at least one camera or camera sensor suitable for detecting corresponding light, in particular infrared light. The illumination device may comprise at least one light source whose emitted light has or consists of wavelengths smaller than that of visible light. In this case, the camera device has at least one camera or camera sensor suitable for detecting corresponding light.

Preferably, at least two light sources are arranged at a distance from each other. This allows the field of view of the camera(s) to be illuminated more homogeneously and the intensity of individual, light direction-dependent reflection areas can be reduced. This is advantageous in particular with regard to downstream digital image processing and evaluation, especially with regard to outline recognition of objects by means of image processing.

Preferably, the illumination device comprises at least one light source that is arranged or fastened or can be arranged/fastened on an underside of a bearing plate. Preferably, the illumination device has at least two or more light sources that are arranged or fastened or can be arranged/fastened at different positions along an underside of a bearing plate.

Preferably, the illumination device comprises at least one optical filter through which the light emitted by the illumination device is partially or completely filtered. The optical filter can be a polarizing filter which, in particular, is matched to a polarizing filter of the camera device, in particular in order to develop the optimum desired filter effect.

Preferably, the illumination device comprises at least one light diffuser, whereby the illumination device emits a diffuse light. A light diffuser can be or comprise, for example, a milky Plexiglas plate. The light diffuser can in particular reduce or prevent hard shadows and reflections, which is particularly advantageous with regard to downstream digital image processing and evaluation.

Preferably, the image capture device, in particular the camera device and/or the illumination device has at least one aperture, preferably an aperture diaphragm, which in particular has variable diameter, e.g. an iris diaphragm, in order to control the light flow of the illumination or the light flow entering the camera.

Preferably, the image capture device, in particular the camera device and/or the illumination device has at least one or more optical lenses.

Preferably, the image capture device, in particular the camera device and/or the illumination device has at least one lens, preferably a wide-angle lens, preferably a wide-angle fisheye lens.

Preferably, the image acquisition device comprises a timer. Preferably, the data processing device is programmed to activate at least one or more light sources of the illumination device in a predetermined temporal sequence and, in particular, to deactivate them again after a predetermined activity time in each case and/or to activate all or more light sources simultaneously, and preferably programmed for the camera device to capture a plurality of images of the storage area, each captured successively and, in particular, synchronously with the activity times of the lighting.

Preferably, the image acquisition system is configured to capture and store the time of entry of an object into the incubator chamber and/or the time of removal of an object from the incubator chamber.

Preferably, the image acquisition system is configured to,
illuminate by means of the illumination device at least one or two objects arranged on this storage area,
capture an image of the at least one or two objects on this storage area by means of the camera device, and
store the image of the at least one or two objects by means of the data processing device in the form of image data in the data storage device. In this way, various uses of the image data are made possible, in particular: distinguishing objects in the storage area, in particular: assigning different identification data to the first and second object; counting the objects; recognizing the object class; analyzing, storing, recognizing individual features; tracking objects when they move; recognizing and storing the time of entry or removal of the object.

Preferably, the data processing device is programmed to,
distinguish, by means of evaluation of the image data, the first object and second object represented in the image, in particular: to assign different identification data to the first and second object; to count the objects; to recognize the object class; to analyze, store, recognize individual features; to track objects in motion, in particular by means of image processing algorithms to detect the outlines of the first and second object in the image, and
in particular information about the first and second objects, in particular the bounding boxes and/or outlines of the first and second objects, in the form of object data in the data storage device.

Preferably, in a preferred embodiment, the illumination device is arranged, and in particular the data processing device is programmed, to operate the illumination device in at least two different illumination modes, and the image acquisition system is preferably arranged, and in particular the data processing device is programmed, to,
the storage area of the incubator chamber by means of the illumination device
i) illuminate first in a first illumination mode and
ii) afterwards in a second illumination mode different from the first one,
capture at least one image of the storage area during illumination by means of both the first and the second illumination mode by means of the camera device, and
provide the at least one image in the form of image data containing combined image information acquired during both the first and second illumination modes, wherein the data processing device is programmed to execute an image analysis program that obtains the combined image information from the image data.

This embodiment may in particular improve or optimize the quality of the image acquisition of the storage area, which is particularly advantageous with regard to downstream image processing, in particular image evaluation, especially for the detection of the bounding box(es) and/or the outline(s) of one or more objects or cell culture containers.

A first illumination mode and a second illumination mode can differ in particular in that different light sources are used, and/or light sources arranged at different positions, and/or different exposure times of the light sources, and/or different emission spectra or light colors, and/or different light intensities. The different illumination modes can in particular improve or optimize the quality of the image acquisition of the storage area, which is advantageous in particular with regard to downstream image processing, in particular image evaluation, in particular for detecting the outline(s) of one or more objects or cell culture containers.

Preferably, the at least one image of the storage area includes at least a first image of the storage area and a different second image of the storage area, wherein the first image is acquired in the first illumination mode and the second image is acquired in the second illumination mode, and the first image is provided in the form of first image data and the second image is provided in the form of second image data, wherein in particular the data processing device and/or the image analysis program are programmed in such a way that
the first image data and the second image data are combined to obtain combined image data, which in particular results from an addition and/or averaging of first and second image data, and
the combined information is obtained from the combined image data.

This embodiment can, in particular, improve or optimize the quality of the image acquisition of the storage area, which is particularly advantageous with regard to downstream image processing, in particular image evaluation, especially for detecting the positions of one or more objects or cell culture containers in the image of the storage area using bounding box algorithms.

A typical program code for image processing object tracking, which is preferably used, is based on the evaluation of the temporal sequence of images. A typical program code for object tracking uses a “bounding box” as output format to identify an object in an image, to define its collision boundaries and, in particular, to localize it. In digital image processing, the “bounding box” refers to the coordinates of the rectangular frame that encloses most or all of an object shown in the digital image. The use of bounding boxes in object tracking makes it more efficient, since image evaluation by means of such a numerical tool requires fewer computational steps and thus less computational power, especially in comparison with algorithms for outline detection of objects. In addition, the corresponding algorithms can be executed efficiently and cost-effectively using specialized graphics processing units (GPUs). Suitable programming interfaces (APIs) for object tracking using bounding boxes are available in the OpenCV program library under the names BOOSTING, CSRT, GOTURN, KCF, MEDIANFLOW, MOSSE, MIL, TLD. Accordingly, program libraries (“MultiTracker”) are available in OpenCV for simultaneous tracking of multiple objects (“multiple object tracking”). Alternatively, deep learning algorithms for multi-object tracking (MOT) according to the “tracking-by-detection” principle are known.

However, it is also possible and preferred that for object tracking a determination of the contour of the object to be tracked in the image is performed, and in particular the separation of object (foreground) and background by background subtraction.

Preferably, a plurality (N>=10) of illumination modes are used to either capture an image or capture multiple images which then provide a combined image with combined image information in the form of combined image data. Preferably, 2<=N<=300, preferably 10<=N<=300, preferably 100<=N<=300, where preferably N<=500 or N<=1000.

Preferably, the at least one image of the storage area includes a multiple exposure image of the storage area, the image acquisition system being particularly configured to,
expose and capture the image of the storage area during illumination by means of both the first and second illumination modes by means of the camera device, and
provide the multiple exposed image in the form of the image data.

Preferably, the at least one image contains information about objects arranged in the storage area, in particular information, optionally,
about the positions of the objects in the storage area
over the outer contours of the objects,
over the area of the objects, measured in a plane parallel to a planar surface of the storage area,
the area of the storage area not occupied by the objects, measured in a plane parallel to a planar surface of the storage area.

Preferably, the illumination device comprises at least a first and a second light source which are operated differently in the first and second illumination mode, wherein in particular the first and a second light source are arranged at a distance above a bearing surface of the bearing area, wherein in particular the first and second light source are arranged offset in a plane which is parallel to a planar bearing surface of the bearing area, wherein in particular the bearing area has a planar bearing surface, wherein the first light source is arranged vertically above a first half of the storage area and the second light source is arranged vertically above a second half of the storage area, wherein in particular the illumination device comprises an LED light strip with several LED light sources, which is arranged in a plane lying parallel to a planar storage area of the storage area, in particular in a meandering course, a spiral-like course, in particular in an at least partially linear course, wherein in particular the image acquisition system comprises an, in particular programmable, electronic control device, which is set up or programmed in such a way that the first light source is arranged in a meandering course, a spiral-like course, in particular in an at least partially linear course, wherein in particular the image acquisition system comprises an, in particular programmable, electronic control device, which is set up or programmed in such a way that
during an illumination phase of the first illumination mode, the first light source is operated differently than during an illumination phase of the second illumination mode, and/or
during an illumination phase of the first illumination mode, the second light source is operated differently than during an illumination phase of the second illumination mode, especially that
during an illumination phase of the first illumination mode, the first light source is active and during an illumination phase of the second illumination mode, the first light source is less active (i.e. emits with lower intensity) or inactive, and/or
during an illumination phase of the first illumination mode, the second light source is less active or inactive and is active during an illumination phase of the second illumination mode, especially that
during an illumination phase of the first illumination mode, the first light source is operated with a different emission spectrum than during an illumination phase of the second illumination mode, and/or
during an illumination phase of the first illumination mode, the second light source is operated with a different emission spectrum than during an illumination phase of the second illumination mode.

Preferably, in particular, the at least one camera is arranged at a distance vertically above a bearing surface of the bearing area, preferably the at least one camera having wide-angle optics, in particular having a wide-angle or fisheye lens, preferably exactly one camera being provided which is arranged at a distance vertically above a center of the bearing surface of the bearing area.

Preferably, the image acquisition system is a modular component of the incubator, namely one that can be optionally inserted by the user, wherein in particular the incubator has a control device and a temperature control device for controlling the temperature in the interior space of the incubator chamber, wherein the image acquisition system has another control device that is arranged to control the image acquisition system, in particular in that this other control device includes the data processing device of the image acquisition system. Such a modular embodiment of incubator with image acquisition system preferably further comprises a data interface to the incubator, so that e.g. image data can be displayed on the incubator display.

Preferably, in particular, the incubator comprises a control device and a temperature control device for controlling the temperature in the interior space of the incubator chamber, wherein in particular this control device is arranged to control the image acquisition system, in particular by this control device including the data processing device of the image acquisition system. This is the integral embodiment of incubator with image acquisition system.

Preferably, the incubator has a display and is preferably set up or programmed to display on the display preferably the image, and/or preferably image information taken from the at least one image, and/or preferably display an image of the storage area containing the combined image information.

Preferably, the image acquisition system:
an object detection system in which the data processing device is programmed to detect the at least one object located in the storage area during image acquisition of the at least one image by means of the image analysis program,
in particular an object recognition system for recognizing the object on the basis of individual properties and/or for recognizing an object class on the basis of object-specific class properties,
in particular an object tracking system for tracking changes in position of the at least one object in the storage area starting from a start position for detecting its end position.

“Down” means the direction of gravity, “up” means the opposite direction. “Vertical” means “along the vector of gravity”, “horizontal” means perpendicular to the vertical or in a planar plane perpendicular to the vertical. In intended use, incubators are arranged so that the tops of the planar bearing plates are horizontal.

Preferably, the incubator comprises a treatment device for treating the at least one object, in particular cell culture container. The term “treatment” means in particular that an object, in particular a cell culture or a cell culture container is moved, and/or transported and/or examined and/or changed, in particular physically, chemically, biochemically or in any other way.

A treatment device may be a movement device by means of which the cell medium in at least one cell culture container is kept in motion, preferably via a movement program controlled by the control program. A movement device may be a shaking or pivoting device. A movement device preferably has a support device, in particular a plate, on which one or more cell culture containers are placed and/or fixed. A movement device preferably has a drive device, in particular in the case of a shaking device for example an oscillator drive, in particular in combination with an eccentric, by means of which the desired movement program is implemented. A treatment device can be a swiveling device by means of which at least one cell culture container is swiveled. The components of the swiveling device can correspond to those of the shaking device, but are set up for a swiveling movement.

A treatment device may also be a transport device by means of which at least one cell culture container can be transported in the incubator chamber.

The transport device may be a lift device comprising a carrier device on which at least one object, in particular a cell culture container, camera, or light source, can be placed. The transport device or lift device preferably has a movement mechanism and/or an electrically controllable drive mechanism for driving the movement mechanism. The transport device may further be a movable and electrically controllable gripping arm for gripping and holding at least one cell culture container. The transport device may include a conveyor belt or rail system for moving the at least one object placed thereon/on it. The transport may move the at least one object in the incubator chamber, in particular to a processing position or pick-up position, e.g. in a processing station, in the incubator chamber, and away from this processing position or pick-up position. The control device can be set up to control the transport device as a function of information from previously acquired image data.

A treatment device may also be a transport device by means of which at least one camera of the camera device and/or at least one light source can be transported in the incubator chamber. In particular, the transport device can be arranged below or directly below a storage plate, and/or below or directly below a ceiling wall of the incubator chamber. Different illumination modes can be realized by a moving or movable light source. In particular, the illumination mode can be adapted to an occupancy state, e.g. in order to variably realize a suitable illumination direction in case of very dense occupancy of a storage area with objects. Using several cameras, as well as a moving or movable camera, different images or image sections of the storage area can be created, which can be combined into an overall image of the storage area, in particular by digital image processing. In the case of a movable camera, it is also possible to adapt the camera position to an occupancy state, e.g. in order to realize a suitable illumination direction when a storage area is very densely occupied with objects.

In particular, the data processing direction may be programmed to transport the at least one camera of the camera device and/or at least one light source in the incubator chamber by means of the transport device in a predetermined or dynamically adapted manner. For example, the data processing direction can be programmed to transport the at least one camera of the camera device and/or at least one light source in the incubator chamber by means of the transport device to different recording positions, in particular to evaluate the image created there in each case by means of an image processing algorithm and in particular to check whether a desired image information, e.g. an individual feature of a cell culture container, in particular a barcode, has been recorded in sufficient quality, e.g. in order to read the barcode unambiguously. The data processing direction may be programmed to move the camera and/or light source to other capture positions until a desired image information has been captured.

The camera and/or illumination device may also be attachable to a transport device. The camera and/or illumination device can be attached or fastened to a positioning mechanism by means of which the camera and/or illumination device can be moved and positioned in the incubator chamber. The positioning mechanism may include a movable robot arm and is preferably electrically controllable, in particular by a control program of the control device. In this way, different recording situations can be successively recorded with one or with a few camera and/or illumination devices. The positioning mechanism can be designed as a component that can be inserted into the incubator chamber. The power supply of this component may be provided via a cable connection to the incubator, preferably through a wall opening, e.g. a port, or via such a cable connection to an external power source. The control device may be arranged to control the positioning mechanism in response to cell monitoring data.

The term treatment device may also be understood to mean the temperature control device of the incubator chamber, which is used to control the atmosphere inside the incubator chamber to the desired value, in particular 37° C. The term tempering refers to raising and lowering the temperature of the atmosphere by heating and cooling. Preferably, the temperature inside is adjusted by changing the temperature of the walls of the incubator. Temperature sensors of the corresponding temperature control device are distributed in at least one position inside and/or outside the incubator chamber, in particular on a wall of the incubator chamber.

Preferably, the incubator comprises a user interface device via which the user can input data to the data processing device or the control device, and/or via which information can be output to the user. Preferably, the incubator or said user interface device is adapted to allow the user to input at least one operating parameter for operating the incubator or the image acquisition system to or receive information from said user interface device. In this way, a single user interface device may be used by the user to influence, or control, or obtain information from, the incubator and also the at least one image acquisition system. In particular, the image acquisition system can be set up to display position data or free storage space to the user in response to a query of the user made by means of the user interface device of the incubator, or to display information derived from position data (e.g. identity of the user who caused the position change), in particular also statistical information, such as frequency and time of the position change of an object (a sample) and/or—in particular in percent—available free storage space, and/or at least one optical image of the at least one object, in particular with or without the storage area. This is advantageous for the user, because based on this information he receives essential information, which on the one hand allows him to plan experiments more precisely—before he carries out an experiment he knows that storage space is available; on the other hand, the change in position of samples, in particular in the first hours after the seeding of adherent cells, negatively influences their adherence; a uniform cell lawn is then not formed. Providing the information on position changes or their frequency according to the invention allows the user to determine causes of non-uniform cell growth and thus to take them into account in future experiments.

A device-controlled treatment of the incubator is preferably a program-controlled treatment, i.e. a treatment controlled by a program. By a program-controlled treatment of an object it is to be understood that the operation of the treatment is essentially carried out by executing a plurality or a plurality of program steps. Preferably, the program-controlled treatment is performed using at least one program parameter, in particular at least one program parameter selected by a user. A parameter selected by a user is also referred to as a user parameter. Preferably, the program-controlled treatment is performed by means of the digital data processing device, which is in particular part of the control device. The data processing device may comprise at least one processor, i.e. a CPU, and/or comprise at least one microprocessor. The program-controlled treatment is preferably controlled and/or carried out according to the instructions of a program, in particular a control program. In particular, in a program-controlled treatment, substantially no user action is required at least after the program parameters required by the user have been acquired. In particular, a device-controlled treatment of the incubator can be performed in dependence on previously acquired image data. Image acquisition by the image acquisition system is in particular a program-controlled treatment, namely imaging of the storage area or object.

The data storage device preferably comprises at least one data memory, which may in particular be a volatile or a non-volatile data memory. The data acquired or received by the incubator can be stored on this at least one data memory, in particular in at least one database, which can be stored in at least one data memory. Such data includes, in particular, at least one or all of the following types of data: image data, still image data, video image data, object data, combined image data, first and second image data, identification data, ID position data, user identification data, user-related ID position data, object identification data, motion history data, class-related ID position data, individual-related ID position data, occupancy status data. The data storage device is preferably a component of the incubator, i.e. in particular arranged in a housing of the incubator. However, it can also be part of an external data processing device with which the incubator or its data processing device communicates.

A program parameter is a variable which may be set in a predetermined manner within a program or subprogram, valid for at least one execution (call) of the program or subprogram. The program parameter is set, e.g. by the user, and controls the program or subprogram and causes a data output depending on this program parameter. In particular, the program parameter and/or the data output by the program influences and/or controls the control of the device, in particular the control of the treatment by means of the at least one treatment device.

A program or program code or computer program code is understood to mean in particular an executable computer program. This is stored in a data memory or on a data storage medium. A program is a sequence of instructions, in particular consisting of declarations and instructions, in order to be able to process and/or solve a certain functionality, task or problem on a digital data processing device. A program is usually present as software to be used with a data processing device. In particular, the program may be present as firmware, in particular in the case of the present invention as firmware of the control device of the incubator or the system. The program is usually present on a data carrier as an executable program file, often in so-called machine code, which is loaded into the main memory of the computer of the data processing device for execution. The program is processed as a sequence of machine, i.e. processor, instructions by the processor(s) of the computer and is thus executed. By “computer program” is understood in particular also the source code of the program, from which in the course of the control of the laboratory device the executable code can arise.

A user interface device may be a component of an incubator, or a module. A user interface device preferably has in each case: a control device for the user interface device; a communication device for establishing a data connection with a laboratory device, in particular an incubator, via an interface device thereof; an input device for detecting user inputs from a user; an output device, in particular a display and/or a display, for outputting information to the user, in particular a touch-sensitive display. In this context, the control device of the user interface device is preferably set up to exchange data with the control device of the incubator via the data connection.

An object is in particular a cell culture container. A cell culture container is in particular transparent. In particular, it is made of plastic, in particular PE or PS, and in particular has a planar base plate which forms the growth surface of the cells. This may have a surface treatment to promote cell adherence. The cell culture container can be closed or provided with a PE cap or gas exchange cap, in particular a lid with optionally included filter. In particular, the cell culture container is stackable. An Eppendorf cell culture bottle is particularly suitable. The object may be a stack of cell culture containers, in particular a stack of Petri dishes or of cell culture bottles.

Preferably, the data processing device is programmed to detect (time-dependent) changes in the appearance (or appearance) of the objects from one or more images, in particular between longer time intervals of minutes, hours or days. In this way, color changes of the cell culture medium or colors in a cell culture container or structures, e.g. droplets, on a cell culture container wall can be detected. Such colors, color changes, or structures may indicate problems with the respective cell culture, e.g., nutrient deficiency, pH changes, or mold, or other contamination. Preferably, the data processing device is programmed to output information to the user or operating personnel via a user interface depending on the detection of the appearance of a cell culture container or these changes in the appearance of the cell culture container and/or to store the data about this detection (in particular: what was detected and when) in a data memory and to keep it available for retrieval.

Image-processing object tracking techniques are well known, for example when used in drones or driver assistance systems for vehicle or person tracking. Object tracking is based on image processing or image analysis of video image data. Such object tracking methods can be implemented with relatively simple means such as suitable cameras and image processing algorithms. The theoretical basics and their use for practical realization of object tracking techniques are well known (e.g.: “Fundamentals of Object Tracking,” S. Challa et al, Cambridge University Press, 2011). Immediately usable image processing algorithms for object tracking are also freely available (OpenCV.org) and well documented. OpenCV (English abbreviation for Open Computer Vision) is a free program library (BSD license) of algorithms for image processing and computer vision. The OpenCV program library also includes functions for tracking multiple objects in real time. The application of object tracking in incubators has not been published yet and represents an innovation.

A typical mode of operation of image-processing object tracking, which is preferably also used in the object tracking system according to the present invention, is based on the evaluation of the temporal sequence of images. A typical object tracking program code uses a “bounding box” as output format to identify an object in an image, to define its collision boundaries and, in particular, to localize it. In digital image processing, the “bounding box” refers to the coordinates of the rectangular frame that encloses most or all of an object shown in the digital image. The use of bounding boxes in object tracking makes it more efficient, since image evaluation by means of such a numerical tool requires fewer computational steps and thus less computational power, especially in comparison with algorithms for outline detection of objects. In addition, the corresponding algorithms can be executed efficiently and cost-effectively using specialized graphics processing units (GPUs). Suitable programming interfaces (APIs) for object tracking using bounding boxes are available in the OpenCV program library under the names BOOSTING, CSRT, GOTURN, KCF, MEDIANFLOW, MOSSE, MIL, TLD. Accordingly, program libraries (“MultiTracker”) are available in OpenCV for simultaneous tracking of multiple objects (“multiple object tracking”). Alternatively, deep learning algorithms for multi-object tracking (MOT) according to the “tracking-by-detection” principle are known.

However, it is also possible and preferred that for object tracking a determination of the contour of the object to be tracked in the image is performed, and in particular the separation of object (foreground) and background by background subtraction.

On the one hand, the performance potential of an object tracking system is based on a reliable automatic identification of an object in the incubator in different typical usage scenarios of an incubator, which are described below. On the other hand, the approach is efficient because no special adaptations are required on the part of the object. In particular, the object does not need to include passive (code, labeling) or active (e.g., a transmitter) identification aids. Rather, the usual objects (cell culture containers, devices, etc.) can be used with the incubator, in particular regardless of manufacturer and external appearance. In particular, the incubator according to the invention is able to distinguish objects with completely identical appearance by tracking.

Possible scenarios when changing occupancy in an incubator include:
I. Setting new objects
II. Removal of objects
III. Door is opened and objects are only moved without removing or resetting one.

Subconditions:
i) Object(s) are moved
ii) Object(s) are not moved
iii) Several objects are set/taken out (sequence).

Assumption: all cell culture containers look the same externally. The question underlying the development of the invention was, in particular, which image-based methods could be considered, and in particular whether still images are sufficient to enable object identification in common usage scenarios of an incubator.

It is first assumed for scenario I. (new object is to be inserted into the incubator chamber) that there is a current still image of the storage area in the incubator chamber taken by a camera placed in the incubator before the incubator door is opened, which does not yet show the new object.

If the new object is placed in the incubator chamber without moving the stock objects (objects already placed and located in the storage area) (case I.ii)), the new object can be identified (unambiguously without any problems) via the next still image (after closing the incubator door). Object tracking is not necessary for case I.ii). The same applies to II.ii): in case of removal of an object, its identification is unambiguously possible from the evaluation of the still images before and after door opening.

If the new object is set and inventory objects are moved in the process (case I.i)), the new object cannot be uniquely identified via the next still image. Location information about the already registered inventory objects is lost. The same applies to the removal of an object and the resulting movement of the inventory objects (case II.i)). For moving (condition i)) the concept of object tracking comes into play.

If several objects (case iii)) are adjusted under condition i), i.e. without moving the stock objects, the new objects can be easily identified by before-after still images, but the information about the order of adjustment is lost. If you want to get this information, you need the object tracking. The same applies to the case of removing several objects in case i)+iii).

Since moving inventory objects is the rule rather than the exception in the operation of an incubator, an evaluation of the before-and-after still images of the storage area is not sufficient in this case.

One question in particular when developing an object tracking system in an incubator is: When is an object identified, i.e., at what time or event is identification data assigned to the object? In most application scenarios (except for a case such as iii)+i), where it may still be important to record the order in which objects are set), it is sufficient to collect this identification data when the new objects have been set and any inventory objects have been moved in the process. This is because the moving of the already registered inventory objects is done under the object tracking measure. In the next freeze frame, i.e. especially when the incubator door is closed, the registration of the new objects can then take place on the basis of the freeze frame. If, however, the registration of the sequence is desired, at the moment when an object enters the field of view of the camera for the first time, i.e. when it appears for the first time in an image recorded by the camera (a start image, which in this case is a video image), this is registered, i.e. an ID number is assigned to the object, and this object is tracked to its end position, the possible moving of stock objects or their removal is preferably also tracked in the process.

In another practical scenario, the storage area (or several storage areas) is assumed to be occupied by one or more objects (stock objects), which are registered on the basis of an initial start image (in this case, for example, a still image). Only the eventual movement of these inventory objects must be tracked here. The movement of newly set objects does not have to be tracked here during the setting process, because their registration can take place again in the next still image. The corresponding presence of these new objects in the video data can thus be ignored. In this scenario, the information about the sequence of setting several objects during a door opening is lost, but this information is also not absolutely necessary.

Accordingly, for a preferred embodiment, the invention proposes to implement object tracking to ensure the correct localization of objects, as needed, in various or all situations.

The data processing device is programmed in particular to assign identification data to the at least one object introduced into the interior space. This means in particular that a new object is detected in the image data (still images or video data) of the camera. In particular, a new object is detected when it is moved into the camera's field of view from the outside. When the object is detected, identification data can be assigned to it on the one hand, and position data on the other. The position data, in particular the start and end position of an object, are determined in particular with reference to an internal coordinate system, which is also used to define the position of the at least one storage area and thus also the position of the at least one object with respect to the at least one storage area. This position information is of particular importance if the position of the at least one object in the at least one storage area or in the incubator chamber is to be graphically illustrated to the user on a display.

Identification data may be or include an identification number, and/or may include an identification code of any character or information. These identification data can be predetermined, randomly generated, or given by a user, especially as long as they are suitable to uniquely distinguish the newly placed object in the incubator chamber from the identification data of the other stock objects. The identification data may also be predetermined and merely selected. The latter case is covered by the term “assign” as well as the re-creation of the identification data.

In particular, the data processing device is programmed to determine the start position of the at least one object from the start image of the storage area. The start image is preferably a still image taken in a still image mode of the camera. It may also be a still image obtained from video data, in particular a video frame. In particular, the data processing device is programmed to determine an enveloping line figure, preferably rectangle, or an enveloping body, or in particular a bounding box of the object, or an outer contour of the object, in the start image and, in particular, to define the object as the object enveloped by the enveloping line figure, in particular by the bounding box or an outer contour.

The data processing device is programmed in particular to determine the changes in position of the at least one object by evaluating the video data. The data processing device is programmed in particular to track the movement of the object defined in the start image by means of the bounding box. In particular, the data processing device is programmed to detect the movement of the image area defined in the start image by means of the bounding box and containing the object by determining the changes in position of this image area from frame to frame. In particular, tracking of the bounding box can be used to determine the image area changing position due to the object movement. Video data contains in particular information, by means of which the single frames characterizing the video (“frames”, which are represented in the case of the representation of the video with certain number per time thus “framerate”) can be reconstructed. In the case of uncompressed video data, the latter may also contain the complete sequence of frame data, where a “set” of frame data represents one frame at a time. In the case of compressed image data, temporal changes of pixels of the camera image may also/only be captured.

The data processing device is programmed in particular to determine the start position of the object in the storage area from the start image. The start position can be determined in particular by the fact that the object, which was previously motionless in a first frame of an image series, shows a change in position of the object in the subsequent frame. The first frame in which the object shows a change in position compared to the previous frames can be defined as the start frame. Since the motion of the object starts at a time T1 and ends at a time T2, an image (still image or video image, also an image obtained from superposed images) acquired before the time T1 can be used as the start image from which the start position of the at least one object is determined.

The data processing device is programmed in particular to determine the final position of the at least one object in the final image of the storage area from the position changes. The end position can be determined in particular by the fact that no more position changes of the object are determined from frame to frame. The first frame in which the object no longer has any change in position compared to the previous frames can be defined as the final image. Since the motion of the object starts at a time T1 and ends at a time T2, an image (still image or video image, also an image obtained from super-positioned images) acquired from time T2 onwards can be used as the final image from which the final position of the at least one object is determined. The end position can be determined in particular by the time at which a closing of the incubator door is detected by means of the door sensor. In particular, the end position can be determined by the fact that an arm or hand of the user leading into the image area is no longer detected. For example, an image can be evaluated to determine whether a section, e.g., in the form of a strip, located in the image border area corresponds to a reference state in which a reference section of the incubator chamber or incubator is completely visible. If this is not the case, it can be concluded that a user is still handling inside the incubator and the object or multiple objects are still being moved, so that in particular the video image acquisition and analysis is to be continued. The end position of the object can be understood as the position where the object no longer shows a change in position after previous changes in position, thus can be determined by the end of the movement of the object. Alternatively or additionally, the end position of the object may be defined to be the position that the object holds when the closing of an incubator door is detected by means of a door sensor. As a result, the end position will be the same in most cases.

The data processing device is preferably programmed to start capturing the start image and/or video data using the camera when a sensor detects activity occurring at the incubator. The sensor may be a motion sensor that detects motion in a detection area located outside the incubator. The sensor may be a touch sensor that detects a touch performed by a user on the incubator, such as a door handle of the incubator. The sensor may be a door opening sensor that detects an opening of an incubator door, in particular an exterior door of the incubator. The sensor may be an exterior camera of the incubator that uses image analysis to detect motion and/or a person in the camera's field of view. The sensor may be a proximity sensor that detects the approach of a person to the incubator, for example by detecting the change in an electric field.

The data processing device is preferably programmed to start the acquisition of the final image and/or stop the acquisition of video data when a sensor detects an activity occurring at the incubator. The sensor may be a door opening sensor, in particular detecting a closing of an incubator door, in particular an exterior door of the incubator. The sensor may be an outside camera of the incubator that uses image analysis to detect the termination of a movement and/or the disappearance of a person in the camera's field of view. The sensor may be a proximity sensor that detects, for example by detecting the change in an electric field, the removal of a person from the incubator.

In particular, the data processing device is programmed to start the acquisition of video data by means of the camera initiated by the opening of an incubator door detected by means of a door sensor. Alternatively or additionally, an initial event sensor, in particular a motion sensor, or proximity sensor, or optical sensor/receiver (e.g. light barrier), or microphone, or acceleration sensor in the incubator door, can be arranged in the incubator, by means of which the approach of an object to the incubator chamber or another initial event can be detected; Trigger for the initiation of the camera and video data can also be a code entry at a door lock of the incubator, which in particular can be performed by the data processing device without using measurement results from one of said sensors; the data processing device can be programmed to start the acquisition of video data based on the data from such a sensor. The data processing device may be programmed to start searching for a new object in the images (frames) available by means of the video data or the still image when the video data and/or a still image is available. Alternatively, video data acquisition can start as soon as a user is identified at the incubator, or at some other predetermined event. Permanent video data acquisition is also possible. In particular, the data processing device is programmed to terminate the acquisition of video image data with the acquisition of the final image or the registration of the absence of a hand/arm in the camera field of view, or based on the results of one of the mentioned sensors (door sensor, motion sensor, etc.).

The data processing device is programmed in particular to assign identification data to the at least one object in the storage area in each case and to determine the position of the at least one object in each case as ID position data and to store it in the data memory. In particular, the data processing device is programmed to store the final position of the at least one object in the storage area as ID position data in the data memory as a function of the identification data of the at least one object. With this step, the incubator “knows” “the object” and its position. Together with other data, it can now output this data to a user, in particular show it on a display of the incubator. Together with data about the owner (definition owner: the user who placed the object in the incubator chamber) of the object or a user of the object (e.g. a user who moved an inventory object of another user), the incubator can store and collect these data sets depending on the identification data of the object. The identification data on the basis of which the position changes were detected need not be identical to the identification data stored as ID position data; what is relevant is that the stored identification data is suitable for uniquely distinguishing the at least one object from other objects or inventory objects. The ID code can therefore theoretically change during image processing.

The assignment of an owner to an object may be accomplished in a variety of ways. Preferably, the data processing device is programmed to register or identify the user placing the object in the incubator, to assign a user identification code to this user, and to store user-related ID position data of the object. For a registration, a biometrics recognition, in particular face recognition, speech recognition, and/or voice recognition, of the user can be performed, in particular by means of an outdoor camera, a retina scanner, or a fingerprint sensor of the incubator. The corresponding registered biometric recognition data, in particular facial recognition data of the user can be stored in the data storage device of the incubator, or in an external data storage device. The user may be identified based on a comparison of captured biometrics recognition data with previously registered biometrics recognition data. As an alternative to biometrics recognition, a user may also be enabled to enter user identification data via a user interface device before, during, or after performing object registration or determining the final position of a tracked object. The user interface device may be a keyboard, a touch screen, and may be part of the incubator or an external device, or a user name/identifier may be enterable via voice input.

An advantage of object tracking is that it can basically be performed without knowledge of individual or class characteristics of the object to be tracked. However, it can also be combined with methods for object detection (and redetection) and/or object class detection or class redetection. This is especially useful when multiple objects are tracked in parallel using object tracking systems.

An object recognition can be designed in particular as object individual recognition and/or as object class recognition. The theoretical principles and their implementation for practical application in object recognition technologies are known (e.g.: “Deep Learning in Object Detection and Recognition,” X. Jiang et al, Springer Singapore, 2019). Algorithms for object recognition in images are widely known and available, e.g. as part of OpenCV (for example OpenCV 3.3, deep neural network (dnn) module).

Individual object recognition is based on the recognition of object-specific features (individual object features), which can be used to recognize the individual object and distinguish it from other individual objects. For example, a cell culture container, e.g. a disposable product, can have subsequently applied individual features, e.g. a barcode or QR code. However, it can also be identifiable by any features that allow it to be distinguished: e.g., a label, a different content, a micro-scratch pattern on the container surface, etc. . . .

Object class recognition relies on knowledge of object class features that are matched during object inspection to assign a class to the object. For example, object class recognition can be used to identify whether an object is a particular type of cell culture flask, a particular type of Petri dish, or a particular type of microtiter plate, possibly taking into account other class features, e.g., manufacturer, year of manufacture, design, etc., as well.

The incubator preferably has an object detection system. The object tracking system is preferably additionally set up for object recognition.

In the case of object individual recognition, a) a data processing device of the object recognition system or the object tracking system is preferably programmed to recognize individual features of at least one object from a still image, the start image, the video data and/or the end image, and b) to store these individual features of the object in the form of object individual data, in particular as a function of identification data. Preferably, the data processing device is programmed to extract object individual characteristics of the at least one object from the start image, the video data and/or the end image, to compare the object individual characteristics with an object individual database and, if the object individual characteristics in the object individual database are associated with an object individual identifier: to identify the object individual identifier of the at least one object; or, if the object individual characteristics in the object individual database are not linked to an object individual identifier: to assign an object individual identifier to the at least one object and to store it in the object individual database, and/or to assign the recognized object individual identifier to the ID position data of the at least one object and to store it as individual-related ID position data. An object individual identifier is preferably different from its identification data; however, the object individual identifier can also preferably be the same as its identification data.

In the case of object class recognition, a data processing device of the object recognition system or the object tracking system is preferably programmed to a) recognize class features of the at least one object in a still image, the start image, the video data and/or the end image, and b) store these class features of the object in the form of object class data, in particular as a function of identification data. Preferably, the data processing device is programmed to recognize object class features of the at least one object in a still image, the start image, the video data and/or the end image, to match the object class features with an object class database (which in particular contains preknown correlations between the object class and object class features) and to recognize the object class of the at least one object, and in particular to assign the recognized object class as object class data to the ID position data of the at least one object and in particular to store it as class-related ID position data.

Preferably, the incubator comprises a user identification device by means of which a user using the incubator is identifiable in terms of user identification data. Preferably, a data processing device of the incubator is programmed to identify a user using the incubator by means of the user identification device and to assign user identification data thereto and to store identification data and/or ID position data in dependence on the user identification data as user-related identification data and/or user-related ID position data in the data memory.

Preferably, the user identification device comprises an external camera, and preferably the user identification device is arranged and/or the data processing device is programmed to perform a facial recognition by means of the external camera, by means of which the user is identified. Preferably, a user database is provided, which is stored on a data storage device, which may be part of the incubator, the user identification device or the object tracking system, or which may be in a data exchange connection with the user identification device or the data processing device, e.g. via an intranet or the internet. Algorithms for face recognition in images are generally known and available, e.g. as part of OpenCV (“FaceRecognizer class”).

The user database may contain a correlation of user identification data and user feature data so that the user or the user's user identification code (user identification data) can be determined based on the determined or read user feature data. The user feature data may include information about users' facial features, or other biometric data, such as fingerprint data or voice recognition data. The user database may include a correlation of user identification data and user identifiers, where the user identifier may be a personal identification code of the user, e.g., a multi-digit string of characters that a user can use to identify himself or herself when entered at a keyboard of the user interface device.

In particular, the external camera can be arranged on, above or next to an incubator door, in particular an external door on the incubator, or attached to it. Preferably, the external camera is an integral part of the incubator or incubator door. However, it can also be connected to the user identification device or the data processing device via a signal connection, in particular via a data exchange connection, which can be wired or wireless. For example, it is possible to connect the external camera to the incubator or its user identification device or the data processing device via a flexible cable, so that the camera can be freely placed on the incubator by the user.

Preferably, the user identification device comprises a user interface device by means of which user identity data can be read. The user interface device may include a keyboard, and/or a touch screen, and or a microphone for voice input or for implementing user identification by means of voice recognition. The user interface device may be arranged to exchange data with an external data processing device (hereinafter also referred to as “external device”). The external device may be a PC, a smartphone, a tablet computer, or another portable computer with a user interface.

The external device may include means to identify and/or authenticate a user. In particular, currently available smartphones include various means for user authentication, especially facial recognition. The external device preferably has software, e.g., an app, programmed to identify and/or authenticate a user, and in particular to send the result of this process to the user identification device of the incubator via the user interface device. Since an external device also often has its own camera, by means of which facial recognition may be implemented, or a fingerprint sensor, or other hardware for user identification and authentication, the corresponding hardware components are dispensable in the case of connection of the incubator to the external device on the incubator.

The user identification device of the incubator may be programmed as part of a control software of the incubator. The incubator preferably has a control device, which may in particular have a data processing device, which may in particular be programmed to include all or some functions of the user identification device, in particular to control the exchange of data with the external device.

Preferably, the user identification device has a user interface device by means of which user identity data can be selected. For this purpose, in particular the user identification device can have a display or a touch screen by means of which a list of possible users can be displayed, e.g. by specifying a name or image of the user. Input means can then be provided, e.g. keys, a keyboard, touchpad, the touch screen, via which the user can make the selection from the list.

The user identification device may be programmed to perform user authentication by password-protecting said reading of user identity data or said selection from the list, so that the user is not considered identified until authentication is successful.

Preferably, the user identification device comprises a reader for reading a code identifying the user, the reader being in particular an RFID reader, a barcode reader, or a QR code reader.

The user identification device or a/their data processing device can be programmed to unlock and/or lock a locked incubator door depending on the user identification, in particular to unlock a locked incubator door if the user has been successfully identified. In this case, this means that the user is also authorized to access the incubator. However, there may also be an additional access right list that the incubator uses to decide whether an identified user has the access right or, if applicable, what type of access right the identified user has. For example, the access right may be limited to certain times, particularly days of the week, times of day, or authorization time periods. If the incubator has multiple incubator doors, the access right may provide that the user has the access right only for a predetermined selection of those incubator doors.

Preferably, the incubator has exactly one—or even several—incubator door(s) for closing the chamber opening. When closed, the incubator door forms in particular a part of the incubator housing, which serves as a thermal insulator of the incubator chamber of the incubator. The incubator door may have a user interface device on its exterior, in particular a display. A data processing device of the incubator or the user interface device may be programmed to display an image of the at least one storage area of the incubator taken by the camera of the incubator.

Preferably, the incubator has a door sensor for detecting the opening or closing of the incubator door. Preferably, the incubator comprises a motion sensor or a proximity sensor for detecting a person approaching the incubator. Preferably, the data processing device is programmed to start the monitoring of the interior space of the incubator, in particular the generation of the video data/still image data, depending on the detection of a door opening of the incubator and/or the approach of a person;
preferably, the data processing device is programmed to start the monitoring of the interior space of the incubator, in particular the generation of the video data/still image data, in particular as a function of the detection of a door opening by a user identified by means of a user identification device;
preferably, the data processing device is programmed to terminate the monitoring of the interior space of the incubator, in particular the generation of the video data, in response to the detection of a door closure of the incubator;
preferably, the data processing device is programmed to determine, by means of the information from the user identification device and the object tracking device, by which user which object has been moved in the interior space, and to store the user identification data of this user together with the object identification data of this object in the data memory.

Preferably, the data processing device is programmed to determine the movement path of the at least one object within the incubator chamber from the start image, the video data and/or the end image and to store it in the form of movement history data in the data memory, in particular to store it in a time-dependent manner. Preferably, the data processing device is programmed to determine a movement history of the at least one object within the incubator chamber from the start image, the video data and/or the end image and to store it in the data memory in the form of movement history data, in particular to store it in a time-dependent manner, preferably with information about the number and/or times of the changes in the status of the door opening (open/closed) of the incubator door determined by means of a door sensor. The movement path preferably contains stored position data of the object, wherein these position data mark the movement path of the object, in particular between a start image and an end image, in particular between a—in particular unmoved or also moved—start position of the object and a—in particular unmoved—end position. The motion history data preferably contains time-dependent stored position data or motion paths, preferably within at least one time period or during the entire stay of this object in the incubator. Motion history data may also include information about the user initiating the position change in the form of user identification data. This is particularly advantageous for objects containing valuable specimens.

Preferably, the incubator has a display (=a screen). The screen is preferably an integral part of the incubator, in particular of the incubator door. However, it can also be located remotely from the incubator and, in particular, can be part of an external device that can be in a data exchange connection with the data processing device of the incubator.

Preferably, the data processing device is programmed to display a graphical rendering of the interior space of the incubator chamber, in particular the at least one storage area, on the display screen. The graphical rendering may include a photograph of the storage area, which may display one or more inventory objects of the incubator. In particular, the storage area may be a storage plate in the incubator or a predetermined section thereof. The photograph may show an image taken with the camera, which may be post-processed. Preferably, this post-processing involves straightening an image taken by the camera that has been distorted. Algorithms for such post-processing are generally known and freely available (for example: OpenCV, “Omnidirectional Camera Calibration”). The distortion can be especially optical and can be due to the use of wide-angle or fisheye optics.

In particular, the graphical rendering may be an abstracted representation of an image or portion of an image captured by the camera. For example, the graphical rendering may be an abstracted stock area shown from a bird's eye view (or other perspective), in particular the graphical representation of a rectangle or the perspective representation of a cuboid. The inventory objects may also be represented in abstracted form, e.g., as rectangular or cuboid graphic image objects. The goal of such a representation is, in particular, to inform the user of the location of the object(s) in the incubator or storage area. This allows the user to quickly access the desired object(s) and minimizes the time the incubator door is open. In the case where differentiation between individual objects contained in a stack of objects is to be enabled, a graphical rendering from a perspective different from the bird's eye view is useful, for example from a lateral perspective, in order to be able to graphically highlight individual objects of a stack.

Preferably, the data processing device is programmed to graphically display where the object identified by the object location data is located in the storage area or interior space of the incubator chamber, or to graphically display where all objects located in the interior space are located.

Preferably, the data processing device is programmed to graphically highlight one or more objects in the display in response to at least one condition parameter. The condition parameter may denote user identification data. Highlighting is possible both in an abstracted display and in a photographic reproduction of an image or image section of the storage area captured by the camera in the display of the incubator.

Preferably, the data processing device is programmed to graphically highlight on the display, in dependence on user identification data of a user (individual user, a user group, or several users), one object or several objects which is assigned to the user as property, namely, for example, by the user-related ID position data containing the user identification data of this user. The owner is the person who has taken care of the object and—in most cases by himself or with the help of an assistant—has placed it into the incubator chamber. Preferably, the data processing device is programmed to determine, based on predetermined user identification data, where the objects assigned to this user identification data by means of the user-related object position data are positioned and, in particular, to highlight these objects graphically.

The condition parameter can also contain information about a time duration or a point in time, e.g. the time duration with which an object was already arranged in the incubator chamber. This allows a user to quickly get an overview of how long one or more objects have already been stored in the incubator chamber, possibly forgotten there by their owner. Or, depending on an event detected by a sensor of the incubator or depending on a schedule that may be stored in the incubator or an external device, the incubator can graphically highlight one or more objects that require the attention of the user or laboratory personnel.

Or the condition parameter may, in case of implementation of object class recognition, contain information about a particular object class. Thus, one or more objects of the same object class (or the object classes differing from it) can be graphically highlighted, for example, to highlight the location of all Petri dishes (and not: cell culture flasks) in the incubator interior space.

Or, in the case of implementing an object individual recognition, the condition parameter may include information about a particular object individual. In this way, an object search based on individual characteristics can be implemented, for example, by having the incubator include means for entering individual characteristics, such as a barcode, QR code, individual label, or photo of the individual object. It can thus graphically highlight the individual object and make it easy to find.

Preferably, the data processing device is programmed to display on the screen a graphical representation of the interior space of the incubator chamber, in particular the at least one storage area, and in particular to graphically display or highlight the free storage space available in the incubator. For example, the storage area may be shown in an abstracted manner and a free storage position (or several available free storage positions) may be graphically highlighted by displaying the corresponding area in, for example, green or white, or a temporally changing (flashing), contrasting color to the background. In this way, the user does not have to spend time searching for a possible free storage location, or creating one by moving inventory objects.

Moreover, similar to the function of a parking attendant, the data processing device may be programmed to plan the occupancy of the interior space of the incubator chamber or of the at least one storage area and, in particular, to optimize the use of the available storage space in this way. To this end, the data processing device may be programmed to take into account predetermined distances between one or more inventory objects to an object to be newly inserted and, in particular, to suggest them to the user by displaying the free storage space accordingly highlighted as available and/or unavailable. According to these examples, the incubator may comprise a computer/software-implemented planning program for occupying the interior space of the incubator, which in particular takes into account the position of at least one object in the interior space (inventory object) and/or in particular the free available storage space, possibly also the times at which the at least one inventory object has been newly placed, or times in the future at which the placement of further objects in the incubator are planned. Such times may be known, in particular, if the incubator is connected to a laboratory information system (LIS) or other (laboratory) data exchange network. The incubator preferably has a timer, a clock, or a timer.

It is possible and particularly preferred that a data processing device of the incubator is programmed to determine an occupancy state of the interior space of the incubator chamber, and/or is preferably programmed to perform one or more of the following steps, in particular to determine an occupancy state of the interior space of the incubator chamber as a function of the ID position data of the at least one object arranged in the interior space, determining an occupancy state of the interior space of the incubator chamber as a function of the class-related ID position data of the at least one object arranged in the interior space, determining an occupancy state of the interior space of the incubator chamber as a function of the individual-related ID position data of the at least one object arranged in the interior space.

An occupancy state of the interior space may be defined by information describing the volume occupied in the interior space by the at least one object, and/or describing the volume not occupied in the interior space by the at least one object, i.e. the free volume, and/or the storage area occupied by the at least one object on at least one storage area or on the total available storage area in the interior space of the incubator chamber, and/or the storage area not occupied by the at least one object, i.e. the free, storage area on at least one storage area or on the total available storage area in the interior space of the incubator chamber, wherein said information may be related to the total interior space volume or the total storage area, respectively, wherein said information may include, for example, the ratio of an unavailable (occupied) or a free (unoccupied) interior space volume to the total volume of the interior space, or wherein said information may include, for example, the ratio of an unavailable (occupied) or a free (unoccupied) storage area to the total storage area in the interior space.

Occupancy state data containing the information about the occupancy state can also contain the ID position data, class-related ID position data and/or individual-related ID position data. In this way, the specification of a spatial resolution of the occupancy is possible, i.e. the specification for the localization of the occupancy in the interior space, or a density distribution of the objects in the interior space.

It is possible and particularly preferred that a data processing device of the incubator is programmed to store information about the occupancy status of the incubator in the form of occupancy status data in a data memory, in particular to transfer it to an external data processing device, in particular a laboratory device, a PC, or a mobile computer, in particular a tablet computer or a smartphone.

It is possible and particularly preferred that a data processing device of the incubator is programmed to display information about the occupancy status of the incubator on a screen of the incubator or of an external data processing device, in particular in dependence on occupancy status data that may be taken from a data memory. The external data processing device may be part of a laboratory instrument, PC, mobile computer, in particular a tablet computer or smartphone.

In test series based on embodiments of the present invention, it was found that the temperature profile over time in the incubator chamber after opening the incubator door due to temperature control depends on the occupancy state of the incubator chamber. If a larger volume of the chamber interior space is occupied by stock objects, there is a smaller free chamber interior space volume that results from the difference of the chamber interior space volume and the occupancy volume occupied by the objects. In this situation, temperature control designed to control the total interior space volume may produce different, undesirable results. Rapid overshooting may occur, which is undesirable, even though the recovery of the target temperature, e.g. 37° C., may be accelerated in the process, i.e. even though the recovery time may be shortened in the process. If several new objects with lower temperatures than the target temperature are reset, the recovery time may also be delayed, but the knowledge of colder, newly set objects can also be used to adjust the temperature control. Temperature control of the temperature inside the incubator chamber depends on control parameters.

Preferably, an electronic control device of the incubator is set up or programmed to operate at least one temperature control device of the incubator, which is arranged to control the temperature of the incubator chamber, with the electrical power Ptemp(t) during temperature control as a function of the time t. In particular, the incubator can be arranged for the temperature control device to be operated by means of pulse width modulation (PWM) of the current. The power is then determined in particular by the duty cycle of the PWM, since the amplitude of the current is preferably constant. In particular, the mentioned variables can be variables of the temperature control, i.e. control parameters.

Preferably, an electronic control device of the incubator is set up or programmed so that the temperature control or the control of the incubator gas supply (e.g. CO₂, N₂, and/or O₂), in particular at least one control parameter, can be adapted as a function of the occupancy state of the incubator. In this way, the influence of objects arranged in the interior space of the incubator chamber on the response behavior of the controlled system can be taken into account. In particular, the recovery time can be reduced in the event of a greater occupancy of the interior space.

The data processing device of the image acquisition system or other system is preferably separate from a first data processing device of the incubator. However, it may also be part of the control device of the incubator (also referred to as “first control device”) that controls functions of the incubator. In particular, the functions of the control device are implemented by electronic circuits. The data processing device of the image acquisition system may comprise at least one CPU, and optionally at least one GPU. A GPU may be provided for image processing or performing deep learning processes. Alternatively to a CPU, or a GPU, the data processing device may also comprise a dedicated chip, e.g. the Nvidia Jetson, for image processing or performing DeepLearning processes, which may preferably be used in object tracking, in particular in possible object classification or object individual recognition. Such dedicated chips can be added to the data processing device as computational accelerators. A GPU is already present on many System on a Chip (SoC) systems (for graphics and video rendering). A Raspberry PI can also have a dedicated GPU unit as part of the SOC.

The object tracking system may comprise a control device, which may be provided separately from the first control device. The terms “control device” and “control device” are used interchangeably in this description. A control device may include a microprocessor, which may include the data processing device. The microprocessor may be of the “Rasberry Pi” type. The control device and/or the data processing device is preferably configured to perform a control procedure, also referred to as control software or control program—in each case related to the incubator and/or the object tracking system. The functions of the incubator and/or the object tracking system and/or the control device and/or the data processing device can be described in method steps. They can be realized as components of the control program, in particular as subroutines of the control program.

In the context of the present invention, a control device generally comprises or is in particular the data processing device, in particular a computing unit (CPU) for processing data and/or a microprocessor. Preferably, the data processing device of the control device of the incubator may also be arranged for controlling the object tracking system.

The data processing device of the image acquisition system is preferably a device located outside the incubator chamber or incubator and in particular optionally separate therefrom, also referred to as an external device or external data processing device. The data processing device and the incubator are preferably in a data connection and are preferably components of a network for data exchange.

The at least one camera of the image acquisition system is preferably connected to the control device or data processing device of the image acquisition system via a cable connection. For this purpose, the incubator chamber has a through opening (port) through which the cable of the cable connection is guided. Preferably, a seal, in particular a silicone seal, is provided to seal the port in order to prevent (as far as possible) any influence on the atmosphere in the incubator. Alternatively, the camera is connected to the control device or data processing device for wireless data exchange, e.g. via Bluetooth or WLAN.

The incubator may comprise a partial housing in which, in particular, at least one control device (of the incubator and/or of the object tracking system) is arranged. The partial housing is preferably arranged at the rear of the incubator, i.e. in particular opposite the incubator door.

The system, the incubator and/or the image acquisition system and/or the data processing device and/or the control device are preferably configured to use the position data of the at least one object or a plurality of objects to form an electronic documentation file in which the positions and/or movement of the objects and/or their residence time and/or the identification data of the user triggering the movement in the incubator are logged and documented. This documentation file is then stored, in particular in a data storage device, and is preferably continuously updated. In this way, “correct” handling of the objects according to standard protocols can be certified if required. On the other hand, deviations from standard protocols can be subsequently identified and/or information correlations can be determined. By collecting such data, the quality of cell-based laboratory work or medical, biological and pharmaceutical procedures can be significantly improved and become more reliable. The reproducibility of cell-based laboratory work can be increased, and deviations from normal characteristics can be detected at an early stage to allow the user to correct or repeat the experiment at an early stage. The documentation file can be provided to the user or an external data processing device by the control device via data exchange. Such documentation is particularly useful in critical applications, e.g., those with a forensic connection or where cells of significant value are cultured.

More particularly, the invention also relates to a live cell culture incubation system comprising a live cell culture incubator comprising:
an incubator chamber for receiving objects, in particular cell culture containers, which comprises opposing inner walls and a chamber opening for the supply and removal of the objects by a user, and which has at least one storage area for storing the objects extending between the opposing inner walls, an incubator door for closing the chamber opening,
an image processing image acquisition system adapted to retrofit the incubator, the image acquisition system comprising data processing device including a data memory, illumination device, and camera means adapted to capture at least one storage area of the incubator chamber,
wherein the image acquisition system is set up, and in particular the data processing device is programmed to,
   by means of the illumination device illuminate the storage area of the incubator that extends between the inner walls,
   capture, by means of the camera device, at least one image of the storage area extending between the inner walls, and
   store the at least one image by means of the data processing device in the form of image data in the data storage device.

The foregoing system is thus based on an incubator which is retrofittable with a retrofittable image acquisition system, such as is an integral part of the incubator in claim 1, wherein the retrofittable image acquisition system must be correspondingly compatible with the “compatible incubator” so designated. “Retrofitting” preferably includes in each case that the camera device can be suitably arranged or fastened in the incubator chamber, that the illumination device can be suitably arranged or fastened in the incubator chamber, that in particular the data processing device with data memory can be suitably arranged or fastened in/on the incubator, that in particular the in particular the camera device and/or the illumination device and/or the data processing device with data memory are or can be connected to a data processing device of an incubator or of an external computer for the purpose of data transmission, in particular the camera device and/or the illumination device and/or the data processing device with data memory are or can be connected to a power supply which can be part of the image acquisition system or of the incubator. It can also be provided, in particular alternatively or additionally, that the data processing device of the image acquisition system is formed by a data processing device of the incubator, in that the camera device and/or the illumination device are or can be connected to a data processing device of the incubator for the purpose of data transmission.

Preferably, a control device of the incubator according to the invention or of the compatible incubator, which in particular can also control the atmospheric parameters in the incubator chamber (temperature, gas partial pressure CO₂, H₂O etc.) or its data processing device is set up or programmed to determine at least one operating parameter of the incubator as a function of data from the image acquisition system, in particular position data or of the end position of at least one object in the storage area, to determine at least one operating parameter of the incubator, in particular a parameter which controls the display of information on a screen of the incubator or a parameter which is displayed on the screen of the incubator. In particular, position data or the final position of at least one object can be displayed on the screen.

Preferably, the system for incubating live cell cultures comprises: an external device separate from the incubator and in data exchange connection with the latter, in particular a user identification device, in particular a mobile user identification device, and in particular a data exchange device by means of which the data processing device can exchange data with the external device, in particular can determine user identification data by means of the user identification device.

The invention also relates to a method for image acquisition in an incubator which is used for incubating live cell cultures and which comprises:
an incubator chamber for receiving objects, in particular cell culture containers, which comprises opposing inner walls and a chamber opening for the feeding and removal of the objects by a user, and which comprises at least one storage area for storing the objects extending between the opposing inner walls,
an incubator door to close the chamber opening,
an image acquisition system comprising
   an illumination device,
   a camera device and
   a data processing device with a data memory,
wherein the method comprises the steps of:
   illuminate the storage area extending between the inner walls by means of the illumination device,
   capture at least one image of the storage area extending between the inner walls during illumination by means of the camera device, and
   store the at least one image in the form of image data in the data storage device.

The invention also relates to an image processing image acquisition system, in particular adapted for retrofitting an incubator, comprising
an illumination device,
at least one camera device and
a data processing device with a data memory,
the image acquisition system is configured to,
by means of the illumination device illuminate the storage area extending between the inner walls,
capture, by means of the camera device, at least one image of the storage area extending between the inner walls, and
store the at least one image by means of the data processing device in the form of image data in the data storage device.

Further preferred embodiments of the method according to the invention can be obtained from the description of the incubator according to the invention and its preferred embodiments. Furthermore, further embodiment options of the invention result from the embodiment examples in the figures. Identical parts of the embodiments are indicated by substantially identical reference signs, unless otherwise described or otherwise apparent from the context. Showing:

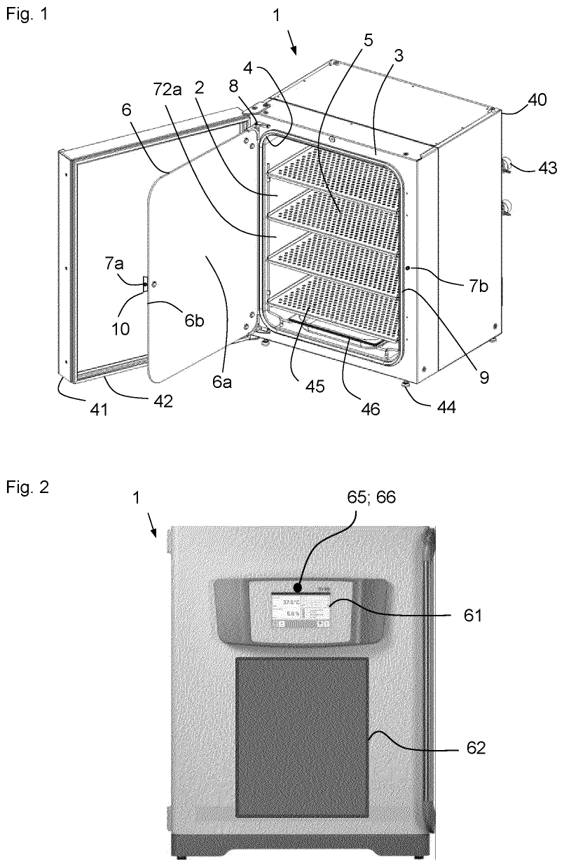

FIG. 1 shows a perspective view of an incubator according to an embodiment of the invention.

FIG. 2 shows a front view of the incubator from FIG. 1.

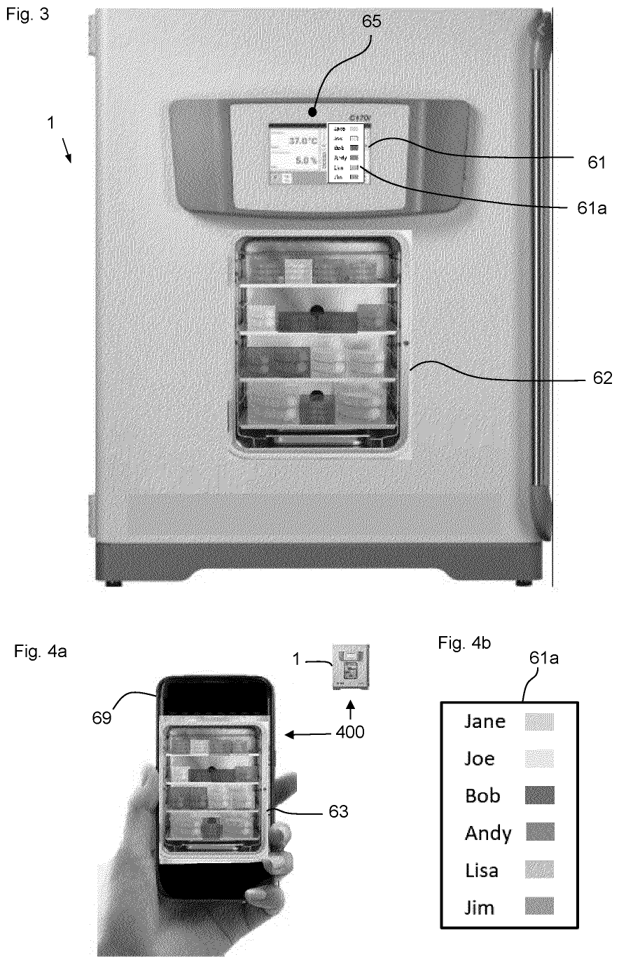

FIG. 3 shows a front view of the incubator from FIG. 1 with a graphic representation of the occupancy of the incubator chamber with objects that are highlighted in a user-specific color-coded manner.

FIG. 4a shows a smartphone with camera and display 63 as an external device that can be part of a system 400 comprising the incubator 1 of FIG. 3 and the smartphone 69.

FIG. 4b shows a legend of the color coding used in the screen of FIG. 3 to highlight user-related objects.

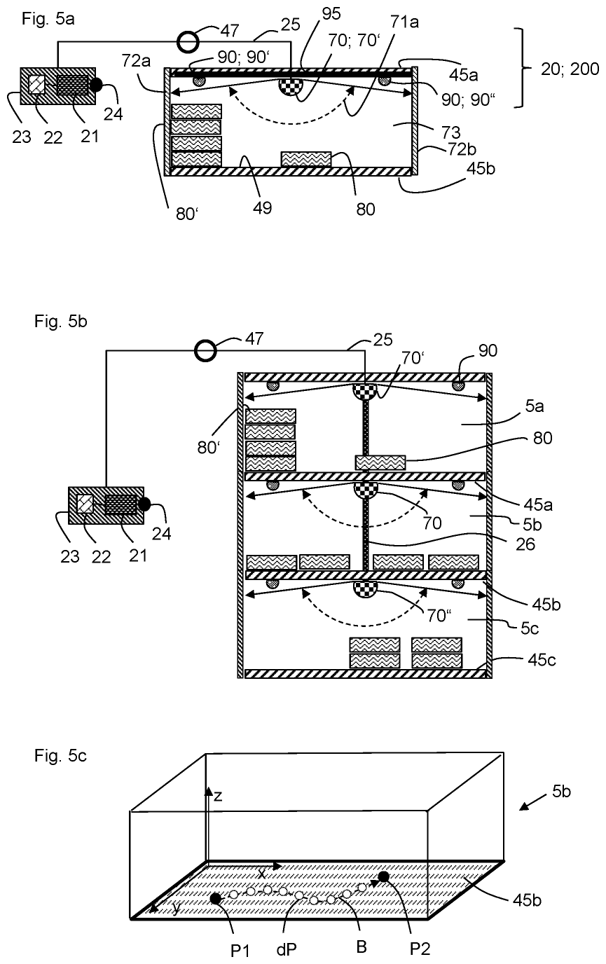

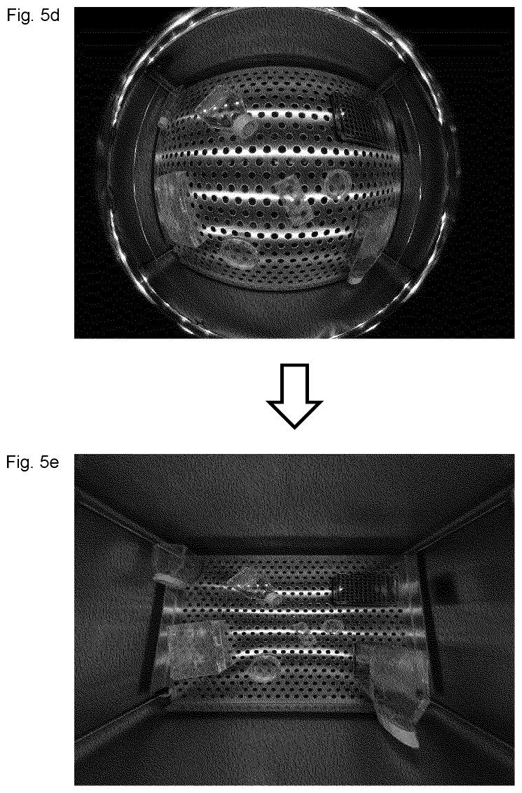

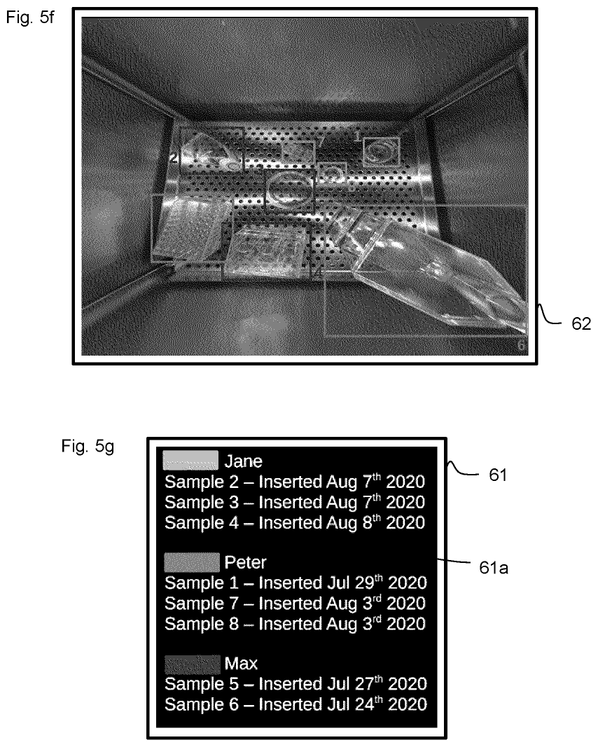

FIG. 5a shows a schematic side view of an image acquisition system as part of the incubator of FIGS. 1 to 4b, in an example of a chamber with a single monitored bearing plate.

FIG. 5b shows a schematic side view of an image acquisition system as part of the incubator of FIGS. 1 to 4b, in an example of a chamber with multiple monitored bearing plates.

FIG. 5c shows a perspective view of a storage area monitored by the object tracking system of FIGS. 5a and 5b, and the start position P1, position changes dP, and end position P2 of a tracked object relative to a coordinate system.

FIG. 5d shows a digital image captured by the wide-angle fisheye camera of the image acquisition system used in FIGS. 5a and 5b, which appears distorted due to the optics.

FIG. 5e shows the image of FIG. 5d rectified by the image acquisition system using straightening algorithms.

FIG. 5f shows a still image captured by the wide-angle fish-eye camera of the image acquisition system used in FIGS. 5a and 5b for output to a screen of the incubator, showing the bounding boxes of the image acquisition system, identification numbers, and color coding identifying the user/owner.

FIG. 5g shows possible screen content that can be displayed on a screen of the incubator to explain the screen shown in FIG. 5f.

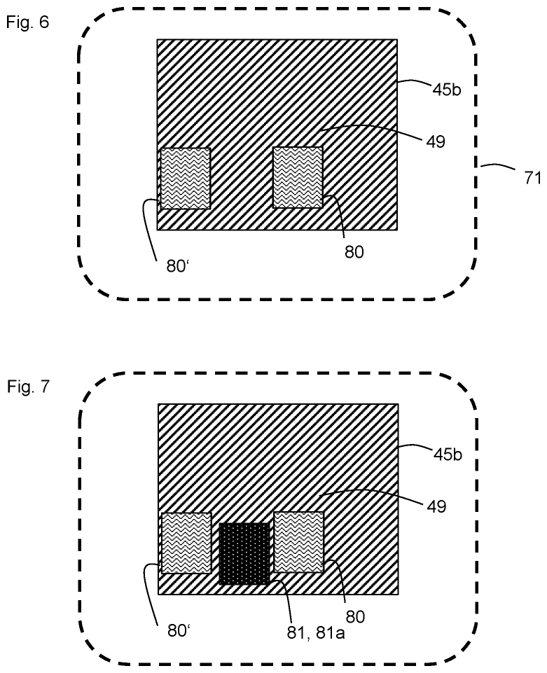

FIG. 6 shows a schematic top view of a storage area of the incubator of FIGS. 1 to 5f, including objects, arranged in an image capture section of a camera of the image acquisition system.

FIG. 7 shows, based on the section of FIG. 6, the detection of an object newly placed in the incubator between two stock objects.

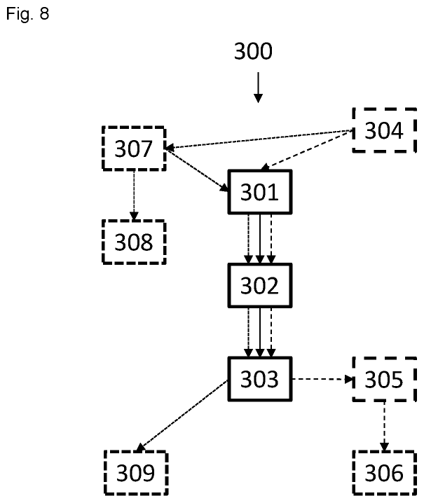

FIG. 8 schematically shows the sequence of an exemplary process according to the invention.

FIG. 1 shows an incubator 1 for storing laboratory samples, more specifically a CO₂ incubator for storing live cell cultures in a defined atmosphere at a controlled temperature, e.g. 37° C. For this purpose, the chamber interior space 5 of the incubator is thermally insulated and can be sealed gas-tight from the environment, and the gas composition in the interior space is also controlled and can be changed via gas connections 43. The chamber housing 2 of the incubator stands on pedestals 44, encapsulates the interior space 5 and opens into the front side 3 of the incubator. The front side has the chamber opening 4 through which the chamber interior space 5 is accessible. A transparent inner chamber door 6 serves to close the chamber opening in a closed position of the chamber door. In the incubator 1, the chamber housing 2 is placed within the interior space of an outer housing so that the chamber housing 2 and the outer housing 40 are spaced apart and thermally insulated from each other. Shelf plate inserts 45 and a humidifier tray 46 are visible in the chamber interior space. The front side 3 of the chamber housing and the front side of the outer housing coincide in the present case.

The outer incubator door 41 and the chamber door 6 are shown in an open position. The outer door 41 is hinged to the outer edge of the outer housing and has a circumferential seal, in particular silicone seal 42.

When the outer door 41 has been opened, the inner chamber door 6 of the incubator is initially still closed. The closing device (10, 7a, 7b) is used for this purpose. With the chamber door 6 closed, the user can first view the interior space 5 through the transparent door wall before opening the door and inserting or removing a laboratory sample. Nevertheless, opening the outer incubator door 41 already represents a disturbance that can potentially damage the incubator atmosphere.

The incubator has an external camera 65 built into the door 41 and facing forwards, the images of which can be analyzed by the suitably programmed data processing device of the incubator, in particular to identify a user by means of facial recognition, whereby the external camera 65 connected to the data processing device serves as a user identification device 66. The latter can also be done via the camera of the smartphone 69.

In the incubator, to protect the stored laboratory samples, it is effective to minimize the time during which the interior space of the incubator is exposed to the environment (opening time intervals). The present invention is based on the observation that the opening time intervals can be reduced by an image acquisition system 200. The incubator 1 has an image acquisition system (not shown in FIGS. 1, 2).

As shown in FIG. 2, the outside of the outer incubator door has a first screen, a touch screen 61, via which operating parameters of the incubator 1 are displayed, e.g. the temperature of the incubator atmosphere or a gas partial pressure in the interior space 5.

The exterior of the outer incubator door 41 includes a second screen 62, which may be a touchscreen. However, instead of a second screen, all screen outputs may be provided on a single screen. The data processing device (not shown) of the incubator 1 is programmed to display on the screen 62 the occupancy of the interior space of the incubator. The screen 62 serves as a “digital window” that allows the user to (virtually) view the interior space of the incubator. In this context, the graphical display of the interior space or the at least one storage area of the incubator and its occupancy by inventory objects can be programmed in such a way that certain inventory objects are graphically highlighted depending on certain criteria or condition parameters.

Re FIG. 3: The data processing device of the incubator 1 is programmed to display one or more objects in the display 62 as a function of at least one condition parameter, which here depends on user identification data, in accordance with their respective position in the interior space of the incubator determined by means of the image acquisition system. In each case, the inventory objects associated with particular user identification data identifying a particular user are highlighted with a particular user-dependent color. The legend 61a for this type of color coding is shown here to the user via the upper display 61 in the sub-area 61a thereof. Legend 61a is shown larger in FIG. 4b: the user identifiers “Jane”, Joe”, etc. are associated with the corresponding highlight colors used in display 62, 63.

In FIG. 4, it is shown that the output display 61 and/or 62 can also—alternatively or additionally—be component(s) of an external device, here a smartphone 69, which is in a data exchange connection with the incubator and has the display 63, which functions here as a component of an incubator system.

FIG. 5a shows in a schematic front view the shelf plate inserts 45a and 45b of the incubator 1, which are arranged one above the other, as storage plates for objects. The vertical distance of such shelf plate inserts 45 in incubators is usually not large and is, for example, between 10 and 40 cm, in the case of incubator 1 about 15 cm. As a result, either several cameras must be used to cover the entire storage area 45, in this case the entire storage area of the shelf insert 45b and the “air space” above it up to the shelf insert 45a. The camera 70 or camera device 70′ is or includes a wide angle or wide angle fisheye optical camera having a diagonally measured angle of view of about 200°.

FIG. 5a shows the image acquisition system 20 installed in the incubator 1, which is also designated by the reference sign 200 in the case of the retrofit system design. The image acquisition system 20 includes the camera 70, a wide-angle fish-eye camera which, in the field of view or angle of view 71a of preferably 160° to 220°, captures the storage area of the shelf plate insert 45b below it and a large portion of about 80% of the areas of the incubator inner wall sections 72a, 72b which, with the storage plates 45a and 45b extending between the inner walls 72a and 72b, define the compartment 73 of the incubator chamber. The wide angle of view makes it possible to get by with a single camera in order to capture the entire storage area of the bottom shelf plate insert 45b, in particular also the air space into which the (stock) objects 80′ and 80 project, namely a stack 80′ of cell culture containers, and a cell culture container 80. The nominal angle of view of the wide angle fish-eye camera is 200° here, but only an image area is evaluated which corresponds to an angle of view taken from the range of preferably 160° to 170°.

The camera is arranged vertically above the geometric center of the storage surface of the shelf sheet insert 45b. The image acquisition system 20 also includes the illumination device 90, the control device 23, which includes a data processing device 21 and a data memory 22 as further components of the image acquisition system 20. The data processing device 21 and the control device 23, respectively, are connected to the camera 70 and the other cameras not shown in FIG. 5a, each of which is provided so that all storage areas (all upper sides of shelf sheet inserts 45, see FIG. 1) are monitored, via a cable connection 25 that enters the incubator chamber through the port 47 in the incubator chamber rear wall. The control device 23 also includes a data interface 24 for enabling a data connection to further incubator device components, for example to output data or signals to a display 61, 62, 63 of the incubator. An illumination device 90 having a plurality of LEDs 90′, 90″ is mounted above the bearing plate 45b and connected to the control device 23 via lines 25. Instead of the two LEDs shown, a plurality of LEDs may be provided. By means of the optional illumination device 90, the bearing area 45b may be illuminated for the purpose of image capture, if appropriate.

FIG. 5b shows a schematic side view of an image acquisition system as a component of the incubator of FIGS. 1 to 4b in an example of a chamber with several monitored bearing plates 45a, 45b, 45c. The embodiment is an extension of the principle of FIG. 5a, in which the incubator chamber is divided into a plurality of compartments 5a, 5b and 5c, which are here arranged one above the other and are connected for gas exchange, which is formed via holes in the bearing plates 45a, 45b, 45c. The bearing area or bearing plate 45a in compartment 5a is monitored by camera 70′, the bearing area or bearing plate 45b in compartment 5b is monitored by camera 70, and the bearing area or bearing plate 45c in compartment 5c is monitored by camera 70″, the cameras 70′ and 70″ being designed and arranged analogously to camera 70 in FIG. 5a. All cameras are connected to the control device 23 via a connection cable bundle 26 inside the incubator chamber, which merges into the cable connection 25 already shown in FIG. 5a and leaves the incubator chamber through port 47 in the incubator chamber rear wall, the data processing device 21 of which is set up to monitor all objects in all three compartments 5a, 5b and 5c. Here, the image acquisition system 20 of the incubator associated with FIG. 5b comprises three cameras 70, 70′, 70″, the illumination device 90, the data processing device 21, the data storage device 22 and the connection lines.

FIG. 5c shows a perspective view of a compartment 5b or storage area 45b monitored by the image acquisition system of FIGS. 5a and 5b, as well as the object positions P1, P2 referred to a Cartesian coordinate system (x, y, z). In the case where the image acquisition system is designed as an object tracking system by acquiring video data and using digital image processing means, position changes dP of an object moving on its motion path B can also be tracked. The origin of the coordinate system may be fixedly located in a corner of the compartment.

FIG. 5d shows a digital image captured by the wide-angle fisheye camera of the image acquisition system used in FIGS. 5a and 5b, which appears distorted due to the optics.

FIG. 5e shows the image of FIG. 5d rectified by the image acquisition system using straightening algorithms.

FIG. 5f shows a still image captured by the wide-angle fish-eye camera of the image acquisition system used in FIGS. 5a and 5b for output to a screen of the incubator, showing the bounding boxes of the image acquisition system, identification numbers, and color coding identifying the user/owner.

FIG. 5g shows possible screen content that can be displayed on a screen of the incubator to explain the screen page shown in FIG. 5f. In addition to identifying the objects by identification numbers, color coding identifying the user/owner is also shown, as well as the optional times registered by the incubator for placing the objects in the incubator chamber.

FIG. 6 shows a storage area, namely the upper side of the shelf plate insert 45b, from a bird's eye view or in top view. Also shown schematically is the image capture section 71 captured by the camera 70. The image capture section 71 is the area captured by the camera 70 in one or each image, because the camera 70 does not change its viewing angle and position here. Thus, each image shows this section 71. In the figures, the lower edge of the section 71 represents the area located near the incubator chamber opening 4. However, the camera 70 and or the illumination device 90 with the two light sources (LEDs) 90′ and 90″ can also be movably or traversably mounted by means of the transport device 95, here a motorized rail system.

The image acquisition system 20, 200 is configured to,
illuminate, by means of the illumination device 90, the storage area 49 extending between the inner walls 72a, 72b,
capture, by means of the camera device 70, at least one image of the storage area 49 extending between the inner walls 72a, 72b, and
store the at least one image by means of the data processing device 21 in the form of image data in the data storage device 22.

The image acquisition system is also configured to,
illuminate, by means of the illumination device 90, at least two objects 80, 80′ arranged on said storage area 49,
capture, by means of the camera device 70, an image of the at least two objects 80, 80′ on said storage area 49—that is, an image on which said at least two objects are shown—, and
store the image of the at least two objects 80, 80′ and the storage area 49 by means of the data processing device 21 in the form of image data in the data storage device 22.

The data processing device is optionally programmed to,
distinguish the first object 80 and second object 80′ represented in the image by means of evaluation of the image data (in particular: to assign different identification data to the first and second object; to count the objects; to recognize the object class; to analyze, store, recognize individual features; to track objects in motion), in particular by means of image processing algorithms to detect the outlines of the first 80 and second object 80′ in the image, and
in particular information about the first 80 and second object 80′, in particular the outlines of the first 80 and second object 80′, in the form of object data in the data storage device 22.

The illumination device 90 is optionally operable herein and the data processing device is optionally programmed herein to operate the illumination device 90 in at least two different illumination modes, and the image acquisition system 20, 200 is configured to,
illuminate the storage area 49 of the incubator chamber by means of the illumination device 90
   initially in a first illumination mode, in particular with active LED 90′ and inactive LED 90″, and
   thereafter illuminate in a second illumination mode different therefrom, in particular with active LED 90″ and inactive LED 90′,
capture at least one image of the storage area 49 during illumination by means of both the first and second illumination modes by means of the camera device 70, and
provide the at least one image in the form of image data comprising combined image information acquired during both the first and second illumination modes, the data processing device being programmed to execute an image analysis program which obtains the combined image information from the image data.

Preferably, the at least one image of the storage area 49 includes at least a first image of the storage area 49 and a different second image of the storage area 49, wherein the first image is acquired in the first illumination mode and the second image is acquired in the second illumination mode, and the first image is provided in the form of first image data and the second image is provided in the form of second image data, wherein the data processing device and the image analysis program are programmed such that
the first image data and the second image data are combined to obtain combined image data, which in particular results from an addition and/or averaging of first and second image data, and
the combined information is obtained from the combined image data.

The data processing device of the image acquisition system 20, 200 is programmed to acquire and evaluate image data by means of the camera 70 during illumination, depending on the detection of the closed state of the outer door 41 of the incubator. By comparing successive images in time, it can be determined whether a new object 81 has entered the camera section 71.

FIG. 7: An image containing a newly appeared outline 81a in the cutout 71, which is assignable to the object 81 placed in the interior space, is regarded as a changed image.

Based on this changed image, identification data is assigned to the newly appeared outline 81a on the assumption that it is a new object 81 to be placed in the incubator.

FIG. 8 shows the sequence of the process according to the invention, which was also indirectly described in the above description of the previous figures.

The method 300 is for capturing images in an incubator, which is used for incubating live cell cultures, and which comprises:
an incubator chamber for receiving objects, in particular cell culture containers, which comprises opposing inner walls and a chamber opening for the feeding and removal of the objects by a user, and which has at least one storage area for storing the objects extending between the opposing inner walls,
an incubator door to close the chamber opening,
an image acquisition system comprising
   an illumination device,
   a camera device and
   a data processing device with a data memory,
   wherein the method 300 comprises the program-controlled steps:
illuminate the storage area extending between the inner walls by means of the illumination device, (301);
capture at least one image of the storage area extending between the inner walls during illumination by means of the camera device, (302) and
store the at least one image in the form of image data in the data storage device, (303).

Preferably also the steps:
monitor, over time, the incubator chamber 2, 5 by means of at least one camera 70 of the camera device 70′ of the incubator arranged to record at least one storage area 49 in the interior space of the incubator chamber into which the at least one object 80; 80′; 81 is placed; (304)
assign identification data to the at least one object 80; 81 captured in an image of the storage area 49 taken by means of the at least one camera 70 after it has been positioned in the storage area; (305)
store the position of the at least one object 80; 81 in the storage area 49 in dependence on the identification data of the at least one object as ID position data, in the data memory. (306)

Preferably, the method 300 also includes the steps of:
read in user identification data identifying the user of the incubator 1 who introduced the at least one object 80; 81 into the incubator chamber by means of a user identification device (307), and
store user identification data in a data memory of the incubator. (308)

Preferably, the method 300 also includes the step of:
store the ID position data as a function of the user identification data as user-related ID position data. (309)

1. An incubator for incubating live cell cultures, comprising
an incubator chamber for receiving objects, in particular cell culture containers, which comprise opposing inner walls and a chamber opening for the feeding and removal of the objects by a user, and which comprise at least one storage area for storing the objects extending between the opposing inner walls,
an incubator door to close the chamber opening,
an image acquisition system comprising
an illumination device,
at least one camera device and
a data processing device with a data storage device,
wherein the image acquisition system is configured to
illuminate the storage area extending between the inner walls by means of the illumination device,
capture by means of the camera device at least one image of the storage area extending between the inner walls, and
store the at least one image by means of the data processing device in the form of image data in the data storage device.

2. The incubator according to claim 1, wherein the image acquisition system is configured to
illuminate at least two objects arranged on this storage area by means of the illumination device,
capture an image of the at least two objects on this storage area by means of the camera device, and
store the image of the at least two objects by means of the data processing device in the form of image data in the data storage device.

3. The incubator according to claim 2, wherein said data processing device is programmed to
distinguish the first object and second object represented in the image by means of evaluation of the image data, to assign different identification data to the first and second object; and
store in particular information about the first and second objects, in particular the bounding box(es) and/or outlines of the first and second objects, in the form of object data in the data storage device.

4. The incubator according to claim 1, wherein the illumination device is adapted to be operated in at least two different illumination modes, and the image acquisition system is configured to
illuminate the storage area of the incubator chamber by means of the illumination device,
first in a first illumination mode and
afterwards in a second illumination mode different from the first one,
capture at least one image of the storage area during illumination by means of both the first and the second illumination mode by means of the camera device, and
provide the at least one image in the form of image data containing combined image information acquired during both the first and second illumination modes,
wherein the data processing device is programmed to execute an image analysis program that obtains the combined image information from the image data.

5. The incubator according to claim 4, wherein the at least one image of the storage area includes at least a first image of the storage area and a different second image of the storage area,
wherein the first image is acquired in the first illumination mode and the second image is acquired in the second illumination mode, and the first image is provided in the form of first image data and the second image is provided in the form of second image data,
wherein the data processing device and the image analysis program are programmed, such that
the first image data and the second image data are combined to obtain combined image data, which in particular results from an addition and/or averaging of first and second image data, and
the combined information is obtained from the combined image data.

6. The incubator according to claim 4, wherein the at least one image of the storage area includes a multiple exposure image of the storage area, the image acquisition system is configured to,
expose and capture the image of the storage area during illumination by means of both the first and second illumination modes by means of the camera device, and
provide the multiple exposed image in the form of the image data.

7. The incubator according to claim 1, wherein the at least one image contains information about objects arranged in the storage area, in particular information, optionally,
about the positions of the objects
about the outer contours of the objects,
over the area of the objects measured in a plane parallel to a planar surface of the storage area,
the area of the storage area not occupied by the objects, measured in a plane parallel to a planar surface of the storage area.

8. The incubator according to claim 1, wherein the illumination device comprises at least a first and a second light source which are operated differently in the first and second illumination mode,
wherein in particular the first and a second light source are arranged at a distance above a bearing surface of the bearing area,
wherein in particular the first and second light sources are offset in a plane parallel to a planar bearing surface of the bearing area,
wherein in particular the bearing area has a planar bearing surface, wherein the first light source is arranged vertically above a first half of the bearing surface and the second light source is arranged vertically above a second half of the bearing surface,
wherein in particular the illumination device has an LED light strip with a plurality of LED light sources, which is arranged in a plane lying parallel to a planar bearing surface of the bearing area, in particular is arranged in a meandering course, a spiral-like course, in particular in an at least sectionally linear course,
wherein in particular the image acquisition system comprises a, in particular programmable, electronic control device which is configured or programmed in such a way that
during an illumination phase of the first illumination mode, the first light source is operated differently than during an illumination phase of the second illumination mode, and/or
during an illumination phase of the first illumination mode, the second light source is operated differently than during an illumination phase of the second illumination mode,
especially that
during an illumination phase of the first illumination mode the first light source is active and during an illumination phase of the second illumination mode it is less active or inactive, and/or
during an illumination phase of the first illumination mode, the second light source is less active or inactive and during an illumination phase of the second illumination mode, it is active,
especially that
during an illumination phase of the first illumination mode, the first light source is operated with a different emission spectrum than during an illumination phase of the second illumination mode, and/or
during an illumination phase of the first illumination mode, the second light source is operated with a different emission spectrum than during an illumination phase of the second illumination mode.

9. The incubator according to claim 1, wherein
in particular the at least one camera is arranged at a distance vertically above a bearing surface of the bearing area,
in particular the at least one camera has wide-angle optics, in particular a fisheye lens,
in particular exactly one camera is provided, which is arranged at a distance vertically above a center of the bearing surface of the bearing area.

10. The incubator according to claim 1, wherein
in particular, the image acquisition system is a modular component of the incubator, namely one that can be optionally used by the user,
wherein in particular the incubator comprises control means and temperature control means for controlling the temperature in the interior space of the incubator chamber, wherein the image acquisition system comprises other control means configured to control the image acquisition system, in particular by said other control means including the data processing device of the image acquisition system,
wherein in particular the incubator comprises a control device and a temperature control device for controlling the temperature in the interior space of the incubator chamber, wherein in particular this control device is adapted to control the image acquisition system, in particular by this control device including the data processing device of the image acquisition system.

11. The incubator according to claim 1, wherein the incubator comprises a display and is configured or programmed to display on the display
preferably the image, and/or
preferably image information taken from the at least one image, and/or
preferably display an image of the storage area containing the combined image information.

12. The incubator according to claim 1, wherein the image acquisition system is an object detection system in that the data processing device is programmed to detect the at least one object located in the storage area during image capture of the at least one image by means of the image analysis program.

13. The incubator according to claim 1, wherein the data processing device is programmed to recognize at least one object stored in the storage area by image processing of the image data, to assign identification data to the at least one object in the storage area in each case, and in particular to determine the position of the at least one object in each case as ID position data and to store it in the data memory, wherein the incubator in particular comprises a display screen and the data processing device is programmed to display a graphical representation of the storage area on the display screen, and in particular to graphically display where the object identified by the ID position data is positioned, respectively to graphically display where all objects located in the storage area or interior space are located.

14. The incubator according to claim 13, wherein the data processing device is programmed to assign user identification data to the ID position data in each case and to store them as user-related ID position data in the data memory, and in particular subsequently, starting from predetermined user identification data, to carry out a comparison with user-related ID position data stored in the data memory in order to determine where the objects assigned to these predetermined user identification data by means of the user-related ID position data are currently positioned in the storage area or in the interior space and, in particular, to mark these objects graphically on the screen.

15. The incubator according to claim 13, wherein the data processing device is programmed to display on the screen free storage space, and/or information derived from the ID position data, in particular the identity of the user who brought about the position change, and/or statistical information, in particular the frequency and time of the position change of an object and/or the percentage of free storage space available.

16. A system for incubation of live cell cultures, comprising
an incubator for incubating live cell cultures, comprising:
an incubator chamber for receiving objects, in particular cell culture containers, which comprises opposing inner walls and a chamber opening for the supply and removal of the objects by a user, and which has at least one storage area for storing the objects extending between the opposing inner walls, an incubator door for closing the chamber opening,
an image processing image acquisition system configured to retrofit the incubator, the image acquisition system comprising data processing device including a data memory, illumination device, and camera means adapted to capture at least one storage area of the incubator chamber,
wherein the image acquisition system is configured, and in particular the data processing device is programmed for this purpose,
to illuminate the storage area of the incubator extending between the inner walls by means of the illumination device,
to capture by means of the camera device at least one image of the storage area extending between the inner walls, and
to store the at least one image by means of the data processing device in the form of image data in the data storage device.

17. A method for image acquisition in an incubator used for incubating live cell cultures, and comprising:
an incubator chamber for receiving objects, in particular cell culture containers, which comprises opposing inner walls and a chamber opening for the feeding and removal of the objects by a user, and which has at least one storage area for storing the objects extending between the opposing inner walls,
an incubator door to close the chamber opening,
an image acquisition system comprising
an illumination device,
a camera device and
a data processing device with a data memory,
wherein the method comprises the steps of:
illuminate the storage area extending between the inner walls by means of the illumination device,
capture at least one image of the storage area extending between the inner walls during illumination by means of the camera device, and
store the at least one image in the form of image data in the data storage device.

18. The method according to claim 17, comprising the steps of:
detect at least one object stored in the storage area using image processing; assign identification data to each object in the storage area;
determine the position of the at least one object in the storage area or interior space as ID position data storing the in the data memory;
display a graphical representation of the storage area on a screen of the incubator, and in particular displaying the information where the object identified by the ID position data is positioned, or where all objects located in the storage area or interior space are located;
in particular visualization of the stored ID position data and/or the user-related ID position data on the screen, in particular by a graphic representation of the interior space of the incubator chamber on the screen, and in particular the graphic display of the information where the object identified by the ID position data is positioned, or where all the objects located in the interior space are located, or display of the free storage space in the interior space of the incubator on the screen.
